SEQUENCE LISTING (5 sequences)

SEQ ID NO: 1 - protein, 17 aa, Artificial Sequence
  Synthetic peptide
KLIPNASLIENCTKAEL

SEQ ID NO: 2 - protein, 15 aa, Artificial Sequence
  Synthetic peptide
ALNNRFQIKGVELKS

SEQ ID NO: 3 - protein, 17 aa, Artificial Sequence
  Synthetic peptide
ISQAVHAAHAEINEAGR

SEQ ID NO: 4 - protein, 11 aa, Artificial Sequence
  Synthetic peptide
CSKKKKKKKKC

SEQ ID NO: 5 - protein, 10 aa, Artificial Sequence
  Synthetic peptide
CKKKKKKKKC

(19) United States
(12) Patent Application Publication
(10) Pub. No.: US 2010/0266623 A1
(43) Pub. Date: Oct. 21, 2010

(54) SYNTHETIC, SELF ADJUVANTING VACCINES

(75) Inventors: David Charles Jackson, North Balwyn Victoria (AU); Weiguang Zeng, Kensington Victoria (AU); Berma Kinsey, Houston, TX (US)

Correspondence Address: KNOBBE MARTENS OLSON & BEAR LLP, 2040 MAIN STREET, FOURTEENTH FLOOR, IRVINE, CA, 92614, US

(73) Assignees: THE UNIVERSITY OF MELBOURNE, Parkville Victoria (AU); BAYLOR COLLEGE OF MEDICINE, Houston, TX (US)

(21) Appl. No.: 12/498,983

(22) Filed: Jul. 7, 2009

Related U.S. Application Data
(60) Provisional application No. 61/078,749, filed on Jul. 7, 2008.

(51) Int. Cl. A61K 39/00; C07K 19/00; A61P 37/04; A61P 25/30

(52) U.S. Cl. 424/185.1; 530/323

(57) ABSTRACT

The present invention relates generally to the field of immunotherapy, and more particularly to immunomedicaments in the form of lipopeptides which induce an antibody response to drugs of dependence, and uses thereof in the treatment and prevention of drug addiction.

### CROSS-REFERENCE TO RELATED APPLICATIONS

This application claims the benefit of U.S. Provisional Application No. 61/078,749, filed Jul. 7, 2008, which is hereby incorporated by reference in its entirety.

### BACKGROUND OF THE INVENTION

1. Field of the Invention

The present invention relates generally to the field of immunotherapy, and more particularly to immunomedicaments in the form of lipopeptides which induce an antibody response to drugs of dependence, and uses thereof in the treatment and prevention of drug addiction.

2. Description of the Related Art

Bibliographic details of references provided in the subject specification are listed at the end of the specification.

Reference to any prior art is not, and should not be taken as an acknowledgment or any form of suggestion that this prior art forms part of the common general knowledge in any country.

Amphetamine-type stimulants (ATS) are a highly addictive class of psychoactive drugs. This group includes various derivatives of amphetamines (AP) such as methamphetamine (MA), also known as “speed” or “crystal”, and 3,4-methylenedioxy-methamphetamine (MDMA), more commonly known as “ecstasy”. MAs are more liphophilic than other psychostimulants, enabling rapid transfer across the blood-brain barrier, leading to a quick psychostimulatory effect.

Other commonly abused drugs include cocaine, nicotine, cannabinods (from marijuana), opiates including morphine and its derivatives, and synthetic pain relievers such as fentanyl and its derivatives. In addition, many of these drugs are administered by injection involving shared needles, leading to the spread of diseases such as hepatitis and HIV, a growing health problem.

Conventional therapy for drug-dependence typically involves counselling, rehabilitation and treatment of associated withdrawal symptoms. However, such therapies have been notoriously unsuccessful, with relapse rates of over 90% reported. Chemical therapy in the form of pharmacological drugs which target the neural pathways involved in addition has been contemplated and in the case of methadone treatment has had some success. However, these drugs result in a range of adverse side effects.

There is a need, therefore, to develop new treatment and preventative protocols for drug dependency.

### SUMMARY OF THE INVENTION

Throughout the specification and claims which follow, unless the context requires otherwise, the word “comprise”, and variations thereof such as “comprises” and “comprising”, will be understood to imply the inclusion of a stated integer or group of integers or steps but the exclusion of any other integer or step or group of integers or steps.

The present invention is predicated in part on the development of immunomedicaments which stimulate the generation of antibodies specific for drugs of dependence. Such immunomedicaments are useful in the treatment and prevention of drug addiction. In particular, the present invention provides a synthetic self-adjuvanting lipopeptide which stimulates the production of antibodies specific for drugs of dependence. The present invention enables a target epitope on a drug of dependence to be exposed, thereby stimulating the humoral immune system via a carrier i.e. the lipopeptide molecule. The terms drug of dependence and drugs of addiction, or like expressions are used interchangeable herein.

Accordingly, in a first aspect the present invention provides a lipopeptide comprising a lipid moiety, a T helper (T_{H}) epitope, a target epitope that is either specific for a drug of dependence or is the drug of dependence and a linker moiety, wherein the linker moiety comprises at least a first, second and third reactive site and wherein the lipid moiety is covalently linked to the first reactive site, the T_{H} epitope is covalently linked to the second reactive site and the target epitope is covalently linked to the third reactive site.

In a second aspect, the present invention provides a lipopeptide comprising a lipid moiety, a T_{H} epitope, a target epitope specific for a drug of dependence or is the drug of dependence and a linker moiety comprising at least a first, second and third reactive site and wherein the T_{H} epitope is covalently linked to the first reactive site, target epitope is covalently linked to the second reactive site and lipid moiety is covalently linked to a third reactive site and wherein the linker moiety is an amino acid or other tri-functional moiety positioned between the T_{H} epitope and target epitope.

In a third aspect, the present invention provides a method of eliciting an antibody response against a drug of dependence in a subject, the method comprising administering to the subject a lipopeptide comprising a lipid moiety, a T_{H} epitope, a target epitope specific for a drug of dependence or is the drug of dependence and a linker moiety, wherein the linker moiety comprises at least a first, second and third reactive site and wherein the lipid moiety is covalently linked to the first reactive site, the T_{H} epitope is covalently linked to the second reactive site and the target epitope is covalently linked to the third reactive site.

In a fourth aspect, the present invention provides a method for treating an addiction to a drug of dependence, the method comprising administering to a subject a lipopeptide comprising a lipid moiety, a T_{H} epitope, a target epitope specific for a drug of dependence or is the drug of dependence and a linker moiety, wherein the linker moiety comprises at least a first, second and third reactive site and wherein the lipid moiety is covalently linked to the first reactive site, the T_{H} epitope is covalently linked to the second reactive site and the target epitope is covalently linked to the third reactive site.

In a fifth aspect, the present invention is directed to the use of a lipopeptide comprising a lipid moiety, a T_{H} epitope, a target epitope specific for a drug of dependence or is the drug of dependence and a linker moiety, wherein the linker moiety comprises at least a first, second and third reactive site and wherein the lipid moiety is covalently linked to the first reactive site, the T_{H} epitope is covalently linked to the second reactive site and the target epitope is covalently linked to the third reactive site in the manufacture of a medicament in the treatment or prevention of drug dependency.

The target epitope on the drug of dependence may be regarded, in one embodiment, as a B cell epitope, referred to herein as a target B cell epitope. The linker is conveniently any entity with at least 3 reactive sites to conveniently link the T_{H} epitope, target epitope and the lipid moiety. In one aspect, the linker is an amino acid or other tri-functional moiety with at least 3 reactive sites. In an embodiment, the amino acid or other tri-functional moiety is located between the T_{H} epitope and the target epitope. In a related aspect, the amino acid is an acidic or basic amino acid. In a particular aspect, the moiety is lysine.

### BRIEF DESCRIPTION OF THE DRAWINGS

FIGS. 1A-D are schematic representations of four possible orientations of lipopeptides of the present invention. In these exemplary lipopeptides the lipid moiety (eg. Pam₂Cys) is attached to the epsilon amino group of the linker moiety (e.g. lysine), and the target epitope and T_{H} epitope are attached to 2 additional reactive sites on the linker moiety.

FIG. 2 is a schematic diagram of the synthesis of the monovalent and divalent DNP-peptide vaccines by Fmoc chemistry. This flow diagram illustrates the stepwise synthesis of totally synthetic small molecule (DNP) vaccines in which either monovalent (left hand path) or divalent (right hand path) vaccines are assembled. Peptide constructs (A) and (C) and lipopeptide constructs (B) and (D) were cleaved from the solid phase support ® by mixing for 2 hours in Reagent B. All constructs were purified by RP-HPLC and characterised by ESI-MS before administration to mice. Details of the syntheses are found in the text.

FIG. 3 is a schematic representation of the synthesis of DNP-BSA. 2,4-Dinitrobenzene sulfonic acid was reacted with BSA in H₂O at pH 9.5 at 37° C. for 48 hours in the absence of light to yield DNP-BSA. DNP-BSA was then purified by FPLC on a Superdex™ 75 column (10 mm×300 mm) and the degree of substitution determined by UV spectrophotometry.

FIG. 4 is a diagrammatic representation of the coupling of succinyl amphetamine to the resin-bound T_{H} epitopes. D-amphetamine was first reacted with a 2 fold molar excess of succinic anhydride in the presence of a 3 fold molar excess of triethylamine for 16 hours at 37° C. to yield succinyl amphetamine which was then coupled to the resin-bound T_{H} epitope in the presence of equimolar amounts of HBTU and HOBt and a 1.5 fold molar excess of DIPEA. The lipopeptide construct was prepared as in FIG. 2.

FIG. 5 is a diagrammatic representation of the coupling of succinyl norcocaine to the resin-bound T_{H} epitopes. The succinyl norcocaine constructs were prepared as described in FIG. 4 for the amphetamine vaccine.

FIG. 6 is a schematic representation of the synthesis of amphetamine-BSA. The carboxyl groups found on the side chains of aspartic acid (Asp) and glutamic acid (Glu) residues present within BSA were activated with N-(3-dimethylaminopropyl)-N′-ethyl-carbodiimide hydrochloride (EDC) to facilitate the coupling of D-amphetamine via its primary amino group to BSA, yielding the protein-carrier based amphetamine construct. Amphetamine-BSA was then extensively dialysed for 24 hours at room temperature against saline to remove unbound amphetamine and free EDC.

FIG. 7 is a schematic representation of the synthesis of cocaine-BSA. The N-hydroixysuccinimide ester of succinyl norcocaine was dissolved in a small volume of acetonitrile before adding to BSA dissolved in PBS. The reaction mixture was adjusted to pH 8 and incubated overnight at 37° C. yielding the protein-carrier-based norcocaine construct. Cocaine-BSA was then extensively dialysed for 24 hours against saline and stored at 4° C. prior to use.

FIG. 8 shows a standard curve for the optical density of DNP at 360 nm produced by UV spectrophotometric analysis of increasing concentrations of 2,4-DNP-glycine. The molar extinction coefficient of DNP is 17,530 M⁻¹cm⁻¹ at λ=360 nm. This graph was used to determine the substitution ratio of 2,4-DNP onto BSA and to estimate the percentage purity of purified and lyophilised DNP-peptides and DNP-lipopeptides which allowed for more accurate calculation of the amount used for immunisations.

FIG. 9 shows analytical RP-HPLC chromatograms representative of purified lipidated and non-lipidated DNP-peptide constructs. The chromatograms were developed at a flow rate of 1 ml/min using the following linear gradients for all non-lipidated peptides: 5 min 0% B, 30 min 40% B, 31 min 100% B, 32 min 0% B, where A=H₂O with 0.1% TFA and B=ACN with 0.1% TFA. Chromatograms for lipidated peptides were developed using the following gradient: 5 min 0% B, 46 min 82% B, 47 min 100% B, 48 min 0% B. Lipidated DNP-T_{H(Flu)} was eluted at 42 minutes (A) and non-lipidated DNP-T_{H(Flu)} at 29 minutes (B). The divalent DNP constructs, lipidated DiDNP-T_{H(Flu)} (C) and non-lipidated DiDNP-T_{H(Flu)} (D) were eluted at 43 and 30 minutes respectively. Chromatography was performed in a Vydac C4 column (4.6 mm×250 mm) installed in a Waters 996 HPLC system.

FIG. 10 shows a comparison between antibody titres of mice sera directed against DNP-constructs. DNP specific antibody titres detected in the sera of groups of 5 female BALB/c mice (A) and C57/B6 mice (B) were observed by direct binding ELISA on DNP₂₀BSA coated plates (1 μg per ml). After subcutaneous (s.c.) delivery of 20 nmol of peptide-based or 100 μg of protein-carrier-based immunogen on days 0 and 21, primary) (1°) and secondary) (2°) sera were collected on day 21 and 31 respectively. Antibody titres were expressed as the reciprocal of the logarithm of the antibody dilution that gave an optical density of 0.2 at 405 nm. The dotted line represents antibody titres that are ≦1. Significant differences in mean antibody titres between 1° (″) sera and 2° ( ( ) sera and groups given different immunogens were computed by one-way ANOVA with a Tukey post-test and represented as a P-value where P>0.05 indicates no significant difference. *, ** and *** represent statistical differences of P<0.05, <0.01 and <0.001 respectively.

FIG. 11 shows a comparison between antibody titres produced against monovalent and divalent DNP-peptide constructs. DNP specific antibody titres detected in the sera of groups of 5 female BALB/c mice (A) and C57/B6 mice (B) were observed by direct binding ELISA on DNP₂₀BSA coated plates (1 μg per ml). Inoculations and serum collections were done as in FIG. 10. Antibody titres were expressed as the reciprocal of the logarithm of the antibody dilution that gave an optical density of 0.2 at 405 nm. The dotted line represents antibody titres that are ≦1. Statistical analysis was done as in FIG. 10.

FIG. 12 shows detection of anti-DNP antibodies in C57BL/6 mice administered non-adjuvanted DNP-T_{H(Ova)} constructs. DNP-specific antibody titres found in the sera of groups of 5 female C57IB6 mice were observed by direct binding ELISA on DNP₂₀BSA coated plates (1 pg per ml). Inoculations and serum collections were done as in FIG. 10. Antibody titres were determined and expressed as the reciprocal of the logarithm of the antibody dilution that gave an optical density of 0.2 at 405 nm. The dotted line represents antibody titres that are ≦1. Statistical analysis was done as in FIG. 10.

FIG. 13 shows a comparison of the antibody titres produced in a DNP-T_{H} mixture study. DNP specific antibody titres found in the sera of groups of 5 female BALB/c mice (A) and C57/B6 mice (B) were observed by direct binding ELISA on DNP₂₀BSA coated plates (1 μg per ml). Inoculations and serum collections were done as in FIG. 10. Antibody titres were determined and expressed as the reciprocal of the logarithm of the antibody dilution that gave an optical density of 0.2 at 405 nm. The dotted line represents antibody titres that are ≦1. Statistical analysis was done as in FIG. 10.

FIG. 14 is a schematic representation of inhibitors used to test for the specificity of anti-DNP antibodies. The ability of DNP-BSA (A), 2,4-DNP-ahx (B), 2,4-DNP-gly (C), 2,4-DNP (D), 2,5-DNP (E) and 2,6-DNP (F) to inhibit antibody binding to DNP₂₀BSA coated plates (1 μg per ml) was assessed in FIG. 15.

FIG. 15 shows the inhibition of 2° sera raised against lipidated DNP-T_{H(MV)} and DNP-BSA administered in CFA To test the specificity of anti-DNP antibodies, limiting dilutions of secondary sera collected from mice immunised with lipidated DNP-T_{H(MV)} (open squares) and DNP-BSA in CFA (closed triangles) were incubated with serial dilutions of various inhibitors before addition to coated ELISA plates. The ability of DNP-BSA (A), 2,4-DNP-ahx (B), 2,4-DNP-gly (C), 2,4-DNP (D), 2,5-DNP (E) and 2,6-DNP (F) to inhibit antibody binding to DNP₂₀BSA coated plates (1 μg per ml) was assessed. Percentage inhibition was determined by comparing with wells lacking inhibitor but identical in all other components of the ELISA system.

FIG. 16 shows analytical RP-HPLC chromatograms representative of purified lipidated and non-lipidated amphetamine- and cocaine-peptide constructs. Analytical chromatograms for peptides were developed at a flow rate of 1 ml/min using the following linear gradients for all non-lipidated peptides: 5 min 0% B, 30 min 40% B, 31 min 100% B, 32 min 0% B, where A=H₂O with 0.1% TFA and B=ACN with 0.1% TFA. Chromatograms for lipidated peptides were developed using the following gradient: 5 min 0% B, 46 min 82% B, 47 min 100% B, 48 min 0% B. Lipidated amphetamine-T_{H(Mv)} was eluted at 44 minutes (A) and non-lipidated amphetamine-T_{H(Mv)} at 27 minutes (B). The cocaine-incorporated constructs, lipidated cocaine-T_{H(mv)} (C) and non-lipidated cocaine-T_{H(Mv)} (D) were eluted at 44 and 29 minutes respectively. Chromatography was performed in a Vydac C4 column (4.6 mm×250 mm) installed in a Waters 996 HPLC system.

FIG. 17 shows a comparison of the ability of different ELISA plate coating antigens to detect anti-amphetamine antibodies. Amphetamine specific antibody titres detected in the sera of groups of 5 female BALB/c mice as observed by direct binding ELISA on amphetamine-BSA (A) and amphetamine-T_{H(ova)} (B) coated plates (1 μg per ml for amphetamine-BSA, 5 μg per ml for amphetamine-T_{H} constructs). After s.c. inoculation with 20 nmol of peptide based or 100 μg of protein-carrier based immunogens at day 0 and 21. 1° and 2° sera were collected on day 21 and 31 respectively. Antibody titre was determined at an O.D of 0.2 and expressed as the reciprocal of log₁₀ with the dotted line representing antibody titres s reciprocal log₁₀ of 1.

FIG. 18 shows the detection of anti-BSA antibodies in the secondary sera of BALB/c mice immunised with amphpetamine-BSA in CFA. Amphetamine specific antibody titres detected in the sera of groups of 5 female BALB/c mice as observed by direct binding ELISA performed on antigen-coated (closed diamonds) or non antigen-coated (open diamonds) plates. 1 μg/ml amphetamine-T_{H(ova)} was used as the antigen coat and all plates were blocked with BSA. After s.c. inoculation with 20 nmol of peptide based or 100 μg of protein-carrier based immunogens at day 0 and 21, secondary) (2°) sera were collected on day 31. Antibody titre was determined at an O.D. of 0.2 and expressed as the reciprocal of log₁₀ with the dotted line representing antibody titres s reciprocal log₁₀ of 1.

FIG. 19 shows inhibition of secondary sera raised against lipidated amphetamine-T_{H(MV)}. To test the specificity of antibodies, limiting dilutions of secondary) (2°) sera collected from mice immunised with lipidated amphetamine-T_{H(MV)} were incubated with serial dilutions of various inhibitors before addition to coated ELISA plates. The ability of amphetamine-T_{H(ova)} (A), amphetamine-T_{H(Flu)} (B), D-amphetamine sulfate (C), to inhibit antibody binding to amphetamine-T_{H(ova)} coated plates (5 μg per ml) was assessed. Percentage inhibition was determined by comparing with wells lacking inhibitor but identical in all other components of the ELISA system.

FIG. 20 shows detection of total and IgA subtype anti-amphetamine antibodies in sera of mice immunised intranasally with lipidated amphetamine-T_{H(MV)}. Amphetamine-specific antibody titres detected in the sera of groups of 5 female BALB/c mice as observed by direct binding ELISA on amphetamine-T_{H(ova)} coated plates (5 μg per ml). After i.n. inoculation with 20 nmoles of peptide based or 100 μg of protein-carrier based immunogens at day 0 and 21, secondary (2°) sera were collected on day 28 and 29 respectively. Antibody titre was determined at an O.D of 0.1 (five times the baseline) and expressed as the reciprocal of log₁₀ with the dotted line representing antibody titres s reciprocal log₁₀ of 0.5.

FIG. 21 shows detection of anti-cocaine antibodies in sera of BALB/c mice after primary and secondary subcutaneous inoculation. Cocaine specific antibody titres detected in the sera of groups of 5 female BALB/c mice as observed by direct binding ELISA cocaine-T_{H(Flu)} coated plates (5 μg per ml). Inoculations and serum collections were done as in FIG. 10. Antibody titre was determined at an O.D of 0.2 and expressed as the reciprocal of log₁₀ with the dotted line representing antibody titres reciprocal log₁₀ of 1. Statistical analysis was done as in FIG. 10.

FIG. 22 is a diagrammatic representation of the synthesis of a morphine-lipopeptide vaccine.

FIG. 23 is a graphical representation demonstrating the generation of morphine specific antibodies in mice inoculated with 6-succinyl-morphine-Lys (Pam₂CysSer₂)-T_{H}.

### DETAILED DESCRIPTION OF THE PREFERRED EMBODIMENT

As used in the subject specification, the singular forms “a”, “an”, and “the” include plural aspects unless the context clearly indicates otherwise. Thus, for example, reference to “a lipopeptide” includes a single lipopeptide, as well as two or more lipopeptides; reference to “an epitope” includes a single epitope or two or more epitopes; reference to “the invention” includes single or multiple aspects of an invention.

The present invention provides lipopeptides which are non-naturally occurring (i.e. synthetic) and which comprise one or more lipid moieties, a peptide sequence comprising at least one T_{H} epitope a moiety comprising a target epitope from a drug of addition, and a linker moiety, wherein the linker moiety comprises at least a first, second and third reactive site and wherein the lipid moiety is covalently linked to the first reactive site, the T_{H} epitope is covalently linked to the second reactive site and the target epitope is covalently linked to the third reactive site. The target epitope may, in one embodiment, be a B cell epitope (i.e. a target B cell epitope).

In a first aspect the present invention provides a lipopeptide comprising a lipid moiety, a T_{H} epitope, a target epitope specific for a drug of dependence and a linker moiety, wherein the linker moiety comprises at least a first, second and third reactive site and wherein the lipid moiety is covalently linked to the first reactive site, the T_{H} epitope is covalently linked to the second reactive site and the target epitope is covalently linked to the third reactive site.

The lipopeptides of the present invention are sufficiently immunogenic such that it is generally not necessary to include an extrinsic adjuvant when being used in the treatment of addiction or as a vaccine. A generalized preferred form of the lipopeptide of the present invention is set forth in Formula (I):

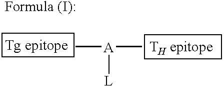

wherein:
Tg epitope is a target epitope from a drug of dependence or a drug of dependence
T_{H} epitope is a T-helper epitope;
A is a linker molecule with at least 3 reactive sites;
L is a lipid moiety, (including a lipoamino acid moiety selected from the group consisting of Pam₂Cys, Pam₃Cys, Ste₂Cys, Lau₂Cys, and Oct₂Cys).

Those skilled in the art will be aware that Ste₂Cys is also known as S-[2,3-bis(stearoyloxy)propyl]cysteine or distearoyl-S-glyceryl-cysteine; that Lau₂Cys is also known as S-[2,3-bis(lauroyloxy)propyl]cysteine or dilauroyl-S-glyceryl-cysteine); and that Oct₂Cys is also known as S-[2,3-bis(octanoyloxy)propyl]cysteine or dioctanoyl-S-glyceryl-cysteine).

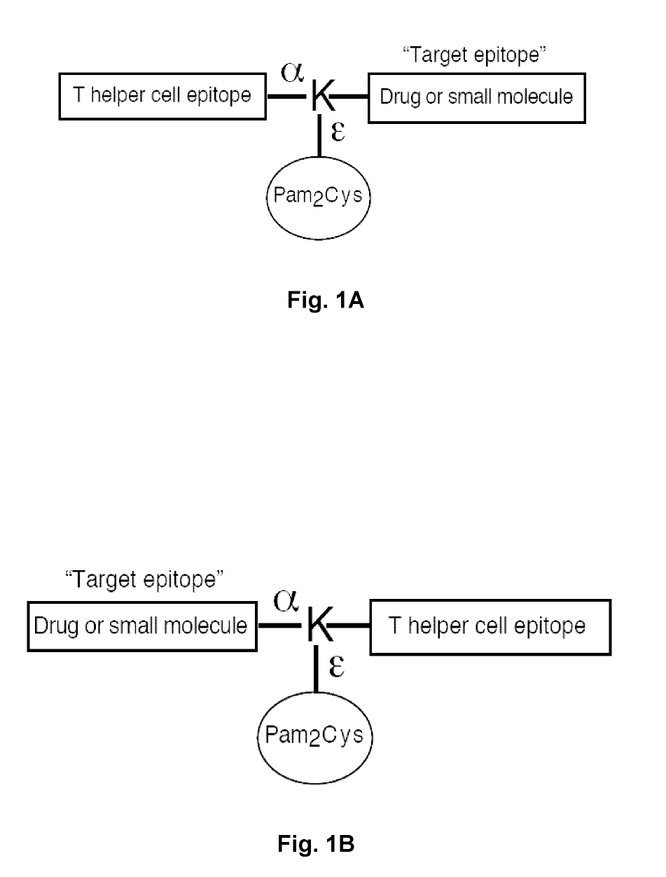

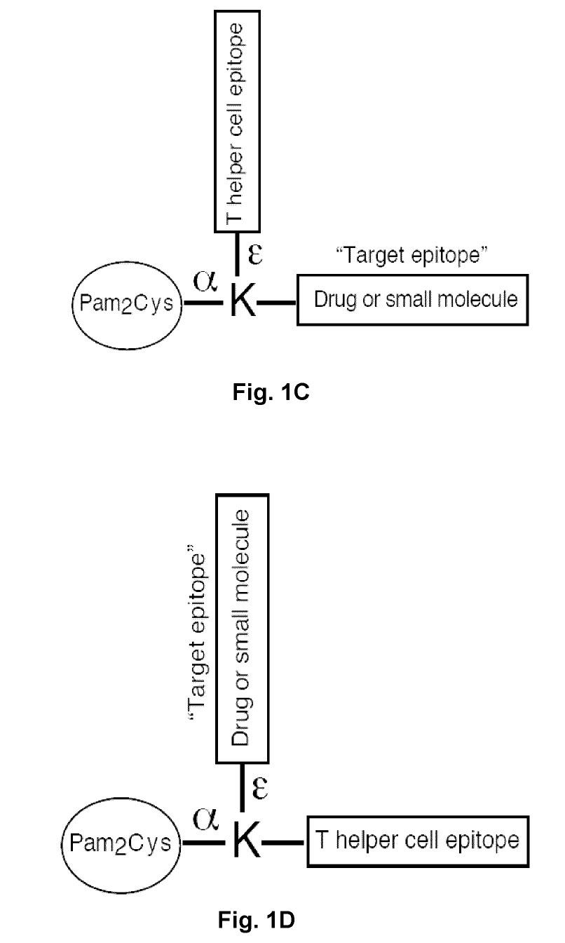

Provides in FIGS. 1A-D are examples of lipopeptides of the present invention. As can be seen from these exemplary lipopeptides the T_{H} epitope, target epitope (or drug) and the lipid moiety (e.g. Pam₂Cys) are all linked to the linker moiety (e.g. lysine).

The lipopeptide comprises a lipid moiety, a T_{H} epitope, a target epitope specific for a drug of dependence and a linker moiety, wherein the linker moiety comprises at least a first, second and third reactive site and wherein the lipid moiety is covalently linked to the first reactive site, the T_{H} epitope is covalently linked to the second reactive site and the target epitope is covalently linked to the third reactive site. In one embodiment, the linker is selected from a basic or acidic amino acid. Some basic amino acids used in the present invention have at least two amino groups, such as lysine, ornithine, diaminopropionic acid or diaminobutyric acid. Acidic amino acids have at least two carboxy groups and include aspartic acid or glutamic acid.

Accordingly, in a second aspect, the present invention provides a lipopeptide comprising a lipid moiety, a T_{H} epitope, a target epitope specific for a drug of dependence and a linker moiety, wherein the linker moiety is an amino acid or other tri-functional moiety between the T_{H} epitope and target epitope.

As an illustration, the linker molecule may be lysine or a lysine analog, such that the amino acid has a suitable side-chain group to which the lipid moiety can be coupled. In one example, the suitable side-chain group is a terminal side chain group.

The term “terminal side-chain group” means a substituent on the side-chain of an amino acid such as a lysine residue that is distal to the alpha-carbon of the residue. The term “amino acid residue” includes an amino acid analog. Hence, examples of terminal side-chain groups include a beta-amino of Dpr, gamma-amino of Dab, or delta-amino of Orn.

As will be known to those skilled in the art, the epsilon amino group of lysine is the terminal amino group of the side chain of this amino acid. Use of the epsilon amino group of lysine or the terminal side-chain group of a lysine analog for cross-linkage to the lipid moiety facilitates the synthesis of the peptide moiety as a co-linear amino acid sequence incorporating the T-helper epitope linked to the target epitope. There is a clear structural distinction between a lipopeptide wherein lipid is attached via the epsilon amino group of a lysine residue or the terminal side-chain group of a lysine analog and a lipopeptide having the lipid attached via an alpha amino group of lysine, since the latter-mentioned lipopeptides can only have the lipid moiety conjugated to an N-terminal residue.

Accordingly, in an embodiment, there is at least one lysine residue or lysine analog to which the lipid moiety is attached. The lysine residue or lysine analog residue may act as a spacer and/or linking residue between the T_{H} epitope and the target epitope. Naturally, wherein the lysine or lysine analog is positioned between the T-helper epitope and the target epitope, the lipid moiety will be attached at a position that is also between these epitopes, albeit forming a branch from the amino acid sequence of the polypeptide. In an embodiment, a single lysine residue or lysine analog is used to separate the T-helper epitopes (e.g., any one of SEQ ID NOs: 1, 2 or 3) and the target epitope, in which case the lipid moiety is attached via the epsilon amino group of a lysine residue or the terminal side-chain group of a lysine analog positioned between the amino acid sequences of the T_{H} epitope and the antigenic B cell epitope.

The epsilon amino group of the internal lysine or the terminal side-chain group of a lysine analog can be protected by chemical groups which are orthogonal to those used to protect the alpha-amino and side-chain functional groups of other amino acids. In this way, the epsilon amino group of lysine or the terminal side-chain group of a lysine analog can be selectively exposed to allow attachment of chemical groups, such as lipid-containing moieties, specifically to the epsilon amino group or the terminal side-chain group as appropriate.

Accordingly in one aspect, the lipopeptide of the present invention comprises a T-helper epitope, a target epitope, a lipid moiety and a lysine linker, wherein the lysine comprises at least a first, second and third reactive site and wherein the lipid moiety is covalently linked to the first reactive site on the lysine, the T_{H} epitope is covalently linked to the second reactive site on the lysine and the target epitope is covalently linked to the third reactive site on the lysine.

The lipid moiety comprises any C₂ to C₃₀ saturated, monounsaturated, or polyunsaturated linear or branched fatty acyl group, or a fatty acid group selected from, but not limited to, the group consisting of: palmitoyl, myristoyl, stearoyl, lauroyl, octanoyl, and decanoyl.

In one aspect, the lipid moieties are covalently linked to the peptide via one of at least 3 reactive sites on a linker. In a related aspect, the lipid moiety is covalently linked via an amino acid or other tri-functional moiety positioned between the T_{H} epitope and target epitope. The lipid moiety may be conjugated to more than one linker or to a residue within the peptide. In a particular aspect, the lipid is linked to a lysine residue. The lipid or fatty acid moiety may also be bound to a post-translationally added chemical entity such as a carbohydrate.

Several different fatty acids are known for use in lipid moieties. Exemplary lipids moieties include, without being limited to, palmitoyl, myristoyl, stearoyl and decanoyl groups or, more generally, any C₂ to C₃₀ saturated, monounsaturated, or polyunsaturated fatty acyl group as is thought to be useful.

An example of a specific fatty acid moiety the lipoamino acid N-palmitoyl-S-[2,3-bis(palmitoyloxy)propyl]cysteine, also known as Pam₃Cys or Pam₃Cys-OH (Wiesmuller et al. Hoppe Seylers Zur Physiol Chem 364:593, 1983) which is a synthetic version of the N-terminal moiety of Braun's lipoprotein that spans the inner and outer membranes of Gram negative bacteria. Pam₃Cys has the structure of Formula (II):

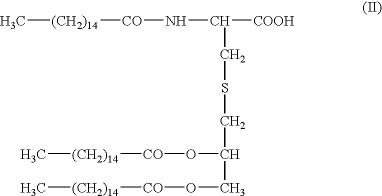

Pam₂Cys (also known as dipalmitoyl-S-glyceryl-cysteine or S-[2,3-bis(palmitoyloxy)propyl]cysteine, an analogue of Pam₃Cys, has been synthesised (Metzger et al. J Pept Sci 1:184, 1995) and been shown to correspond to the lipid moiety of MALP-2, a macrophage-activating lipopeptide isolated from mycoplasma (Sacht et al. Eur J Immunol 28:4207, 1998; Muhlradt et al. Infect Immun 66:4804, 1998; Muhlradt et al. J Exp Med 185:1951, 1997). Pam₂Cys has the structure of Formula (III):

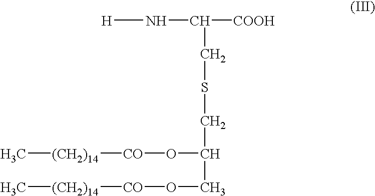

The lipid moiety of the present invention may be directly or indirectly attached to the linker molecule meaning that they are either juxtaposed in the self-adjuvanting immunogenic molecule (i.e. they are not separated by a spacer molecule) or separated by a spacer comprising one or more carbon-containing molecules, such as, for example, one or more amino acid residues.

The lipid moiety, in one aspect, is preferably a compound having a structure of general Formula (IV):

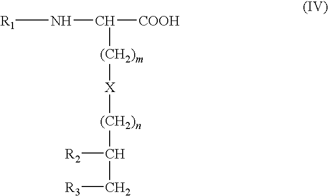

wherein:
(i) X is selected from the group consisting of sulfur, oxygen, disulfide (—S—S—), and methylene (—CH₂—), and amino (—NH—);
(ii) m is an integer being 1 or 2;
(iii) n is an integer from 0 to 5;
(iv) R₁ is selected from the group consisting of hydrogen, carbonyl (—CO—), and R′—CO— wherein R′ is selected from the group consisting of alkyl having 7 to 25 carbon atoms, alkenyl having 7 to 25 carbon atoms, and alkynyl having 7 to 25 carbon atoms, wherein said alkyl, alkenyl or alkynyl group is optionally substituted by a hydroxyl, amino, oxo, acyl, or cycloalkyl group;
(v) R₂ is selected from the group consisting of R—CO—O—, R—O—, R—O—CO—, R′—NH—CO—, and R—CO—NH—, wherein R′ is selected from the group consisting of alkyl having 7 to 25 carbon atoms, alkenyl having 7 to 25 carbon atoms, and alkynyl having 7 to 25 carbon atoms, wherein said alkyl, alkenyl or alkynyl group is optionally substituted by a hydroxyl, amino, oxo, acyl, or cycloalkyl group; and
(vi) R₃ is selected from the group consisting of R—CO—O—, R′—O—, R′—O—CO—, R′—NH—CO—, and R—CO—NH—, wherein R′ is selected from the group consisting of alkyl having 7 to 25 carbon atoms, alkenyl having 7 to 25 carbon atoms, and alkynyl having 7 to 25 carbon atoms, wherein said alkyl, alkenyl or alkynyl group is optionally substituted by a hydroxyl, amino, oxo, acyl, or cycloalkyl group
   and wherein each of R₁, R₂ and R₃ is the same or different.

Depending upon the substituent, the lipid moiety of general Formula (IV) may be a chiral molecule, wherein the carbon atoms directly or indirectly covalently bound to integers R₁ and R₂ are asymmetric dextrorotatory or levorotatory (i. e. an R or S) configuration.

Alternatively, the lipid molecule may be cis or trans from the alkyl group.

In one embodiment, X is sulfur; m and n are both 1; R₁ is selected from the group consisting of hydrogen, and R′—CO—, wherein R′ is an alkyl group having 7 to 25 carbon atoms; and R₂ and R₃ are selected from the group consisting of R′—CO—O—, R′—O—, R′—O—CO—, R′—NH—CO—, and R—CO—NH—, wherein R′ is an alkyl group having 7 to 25 carbon atoms.

In a particular embodiment, R′ is selected from the group consisting of: palmitoyl, myristoyl, stearyl and decanol. In one aspect, R is palmitoyl.

Each integer R1 in the lipid moiety may be the same or different.

In a particular embodiment, X is sulfur; m and n are both 1; R₁ is hydrogen or R′—CO— wherein R is palmitoyl; and R₂ and R₃ are each R′—CO—O— wherein R is palmitoyl. These compounds are shown by Formula (II) and Formula (III) supra.

Amphipathic molecules, particularly those having a hydrophobicity not exceeding the hydrophobicity of Pam3CyS (Formula (II)) are also contemplated. The lipid moieties of Formula (II), Formula (III) or Formula (IV) are further modified during synthesis or post-synthetically, by the addition of one or more spacer molecules, preferably a spacer that comprises carbon, and more preferably one or more amino acid residues. These are conveniently added to the lipid structure via the terminal carboxy group in a conventional condensation, addition, substitution, or oxidation reaction. The effect of such spacer molecules is to separate the lipid moiety from the polypeptide moiety and increase immunogenicity of the lipopeptide product.

Serine dimers, trimers, tetramers, etc, can be used for this purpose.

Exemplary modified lipoamino acids produced according to this embodiment are presented as Formulae (V) and (VI), which are readily derived from Formulae (II) and (III), respectively by the addition of a serine homodimer. As exemplified herein, Pam₃Cys of Formula (II), or Pam₂Cys of Formula (III) is conveniently synthesized as the lipoamino acids Pam₃Cys-Ser-Ser of Formula (V), or Pam₂Cys-Ser-Ser of Formula (VI) for this purpose.

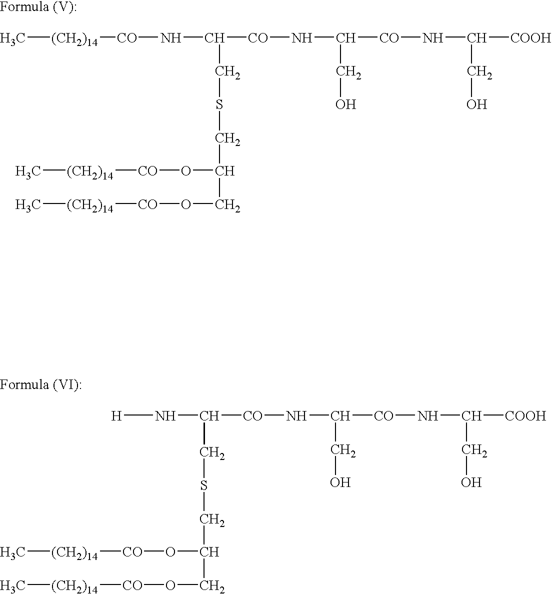

The lipid moiety is prepared by conventional synthetic means, such as, for example, the methods described in U.S. Pat. Nos. 5,700,910 and 6,024,964, or alternatively, the method described by Wiesmuller et al. 1983 supra, Zeng et al. J Pept Sci 2:66, 1996; Jones et al. Xenobiotica 5:155, 1975; or Metzger et al. Int J Pept Protein Res 55:545, 1991). Those skilled in the art will be readily able to modify such methods to achieve the synthesis of a desired lipid for use conjugation to a polypeptide.

Other functional groups such as sulfhydryl, aminooxyacetyl, aldehyde may be introduced into the lipid moieties to enable the lipid moieties to couple to the naturally occurring or recombinant proteins more specifically.

Combinations of different lipids are also contemplated for use in the self-adjuvanting lipopeptides of the invention. For example, one or two myristoyl-containing lipids or lipoamino acids are attached via lysineresidues to the polypeptide moiety, optionally separated from the polypeptide by a spacer, with one or two palmitoyl-containing lipid or lipoamino acid molecules attached to carboxy terminal lysine amino acid residues. Other combinations are not excluded.

The lipid moiety may comprise any C₂ to C₃₀ saturated, monounsaturated, or polyunsaturated linear or branched fatty acyl group, and preferably a fatty acid group selected from the group consisting of: palmitoyl, myristoyl, stearoyl, lauroyl, octanoyl and decanol. Lipoamino acids are particularly preferred lipid moieties within the present context. As used herein, the term “lipoamino acid” refers to a molecule comprising one or two or three or more lipids covalently attached to an amino acid residue, such as, for example, cysteine or serine, lysine or an analog thereof. In a particularly preferred embodiment, the lipoamino acid comprises cysteine and optionally, one or two or more serine residues.

The structure of the lipid moiety is not essential to activity of the resulting self-adjuvanting lipopeptide and, as exemplified herein, palmitic acid and/or cholesterol and/or Pam₁CyS and/or Pam₂Cys and/or Pam₃Cys can be used. The present invention clearly contemplates a range of other lipid moieties for use in the self-adjuvanting immunogenic molecules without loss of immunogenicity. Accordingly, the present invention is not to be limited by the structure of the lipid moiety, unless specified otherwise, or the context requires otherwise.

Similarly, the present invention is not to be limited by a requirement for a single lipid moiety unless specified otherwise or the context requires otherwise. The addition of multiple lipid moieties to the naturally occurring or recombinant polypeptide, for example, to a position within an epitope or to a position between two epitopes is contemplated.

The lipopeptides of the present invention are lipidated by methods well known in the art. Standard condensation, addition, substitution or oxidation The bifunctional linkers described in Pierce Catalogue and the methods therein may liberally be used here. As described in the examples, heterobifunctional linkers, MCS (N-Succinimidyl 6-maleimidocaproate) and SPDP (N-Succinimidyl 3-[2-pyridyldithio]propionate]) were used. In the case of using MCS as heterobifunctional linker, a cysteine residue was incorporated in the lipid moiety Pam₂Cys-Ser-(Lys)₈-Cys (SEQ ID NO: 4) which was coupled to the MCS-modified protein by forming a thioether bond. Pam₂Cys (Lys)₈-Cys (SEQ ID NO: 5) was also coupled to the SPDP modified protein by forming a disulfide bond.

Bromoacetyl or chloroacetyl group may also be introduced into the lipid moieties. These two functional groups can be coupled to the sulfhydryl groups existing or being introduced in the proteins by forming a thioether bond.

Another method involves the incorporation of a serine residue in the N-terminal position of the polypeptide using recombinant or enzymatic or chemical method which is then oxidised to generate an aldehyde function. An aminooxy functional group incorporated in the lipid moiety will form an oxime bond to generate the self-adjuvanting lipid protein.

The other chemical ligation methods such as orthogonal ligation strategies (Tarn et al. Biopolymers (Peptide Science) 51:311-332, 1999), native chemical ligation (Dawson et al. Science 266:243-247, 1994) expressed protein ligation (Muir et al. Proc Natl Acad Sci USA 95:6705-6710, 1998) may also be used to attached the lipid moiety to the polypeptide of the present invention.

As exemplified herein, highly self-adjuvanting immunogenic lipopeptide molecules capable of inducing T_{H} and/or B cell responses are provided, wherein the self-adjuvanting immunogenic molecule in one aspect comprises Pam₃Cys of Formula (II), or Pam₂Cys of Formula (III) conjugated to the peptide.

The enhanced ability of the self-adjuvanting immunogenic lipopeptides of the present invention to elicit an immune response is reflected by their ability to upregulate the surface expression of MHC class II molecules on immature dendritic cells (DC). In an embodiment, the self-adjuvanting immunogenic lipopeptides are soluble.

Effective lipopeptides are those which are highly soluble. The relative ability of the lipopeptides of the invention to induce an antibody response in the absence of external adjuvant was reflected by their ability to upregulate the surface expression of MHC class II molecules on immature dendritic cells (DC), particularly D1 cells as described by Winzler et al J Exp Med 185, 317, 1997.

In one aspect, the present invention discloses the addition of multiple lipid moieties to the peptide.

The positioning of the lipid moiety should be selected such that the association of the lipid moiety does not interfere with the T_{H} or target epitope in such a way as to limit their ability to elicit an immune response. For example, depending on the selection of lipid moiety, the attachment within an epitope may sterically hinder the presentation of the epitope.

As used herein, a T_{H} epitope is any T_{H} epitope which enhances an immune response in a particular target subject (i.e. a human subject, or a specific non-human animal subject such as, for example, a rat, mouse, guinea pig, dog, horse, pig, or goat). T_{H} epitopes comprise at least about 10-24 amino acids in length, more generally about 15 to about 20 amino acids in length.

Promiscuous or permissive T_{H} epitopes are contemplated as these are readily synthesized chemically and obviate the need to use longer polypeptides comprising multiple T_{H} epitopes. In related aspects, the T_{H} epitopes selected are those which are able to generate responses across a broad range of HLA types.

Examples of promiscuous or permissive T_{H} epitopes suitable for use in the lipopeptides of the present invention are selected from the group consisting of:
(i) a rodent or human T_{H} epitope of tetanus toxoid peptide (TTP), such as, for example amino acids 830-843 of TTP (Panina-Bordignon et al. Eur J Immun 19: 2237-2242, 1989);
(ii) a rodent or human T_{H} epitope of Plasmodium falciparum pfg27;
(iii) a rodent or human T_{H} epitope of lactate dehydrogenase;
(iv) a rodent or human T_{H} epitope of the envelope protein of HIV or H1Vgp120 (Berzofsky et al. J Clin Invest 88:876-884, 1991);
(v) a synthetic human T_{H} epitope (PADRE) predicted from the amino acid sequence of known anchor proteins (Alexander et al. Immunity 1:751-761, 1994);
(vi) a rodent or human T_{H} epitope of measles virus fusion protein (MV-F; Muller et al. Mol Immunol 32:37-47, 1995; Partidos et al. J Gen Virol 71:2099-2105, 1990);
(vii) a T_{H} epitope comprising at least about 10 amino acid residues of canine distemper virus fusion protein (CDV-F) such as, for example, from amino acid positions 148-283 of CDV-F (Ghosh et al. Immunol 104:58-66, 2001; International Patent Publication No. WO 00/46390);
(viii) a human T_{H} epitope derived from the peptide sequence of extracellular tandem repeat domain of MUC1 mucin (US Patent Application No. 0020018806);
(ix) a rodent or human T_{H} epitope of influenza virus haemagglutinin (IV-H) (Jackson et al. Virol 198:613-623, 1994);
(x) a bovine or camel T_{H} epitope of the VP3 protein of foot and mouth disease virus (FMDV-0₁ Kaufbeuren strain), comprising residues 173 to 176 of VP3 or the corresponding amino acids of another strain of FMDV;
(xi) T_{H} epitopes from the fusion protein of the morbillivirus and canine distemper virus (T_{H(MV)});
(xii) T_{H} epitopes from the alpha chain of haemagglutinin of Mem71 influenza virus (T_{H(Flu)}); and
(xiii) T_{H} epitopes from chicken ovalbumin (T_{H(ova)}).

As will be known to those skilled in the art, a T_{H} epitope may be recognized by one or more mammals of different species. Accordingly, the designation of any T_{H} epitope herein is not to be considered restrictive with respect to the immune system of the species in which the epitope is recognised. For example, a rodent T_{H} epitope can be recognized by the immune system of a mouse, rat, rabbit, guinea pig, or other rodent, or a human or dog.

Usefully, the T_{H} epitope comprises an amino acid sequence selected from the group consisting of:

| | | |
|---|---|---|
| 1. (T_{H(MV)}): | KLIPNASLIENCTKAEL; | (SEQ ID NO: 1) |
| 2. (T_{H(FLU)}): | ALNNRFQIKGVELKS; | (SEQ ID NO: 2) |
| and | | |
| 3. (T_{H(ova)}): | ISQAVHAAHAEINEAGR. | (SEQ ID NO: 3) |

The T_{H} epitopes disclosed herein are included for the purposes of exemplification only. Using standard peptide synthesis techniques known to the skilled artisan, the T_{H} epitopes referred to herein are readily substituted for a different T_{H} epitope to adapt the lipopeptide of the invention for use in a different species. Accordingly, additional T_{H} epitopes known to the skilled person to be useful in eliciting or enhancing an immune response in a target species are not to be excluded.

Additional T_{H} epitopes may be identified by a detailed analysis, using in vitro T-cell stimulation techniques of component proteins, protein fragments and peptides to identify appropriate sequences (Goodman and Sercarz Ann Rev Immunol 1:465, 1983; Berzofsky, In: “The Year in Immunology, Vol. 2” page 151, Karger, Basel, 1986; and Livingstone and Fathman Ann Rev Immunol 5:477, 1987).

The peptides may be synthesized by a range of techniques including Fmoc and Boc chemistry. For peptide syntheses using Fmoc chemistry, a suitable orthogonally protected epsilon group of lysine is provided by the modified amino acid residue Fmoc-Lys(Mtt)-OH (NI-Fmoc-NM-4-methyltrityl-L-lysine). Similar suitable orthogonally-protected side-chain groups are available for various lysine analogs contemplated herein, eg. Fmoc-Orn(Mtt)-OH (Nα-Fmoc-Nδ-4-methyltrityl-L-Ornithine), Fmoc-Dab(Mtt)-OH (Nα-Fmoc-Nγ-4-methyltrityl-L-diaminobutyric acid) and Fmoc-Dpr(Mtt)-OH (Nα-Fmoc-Nβ-4-methyltrityl-L-diaminopropionic acid). The side-chain protecting group Mtt is stable to conditions under which the Fmoc group present on the alpha amino group of lysine or a lysine analog is removed but can be selectively removed with 1% trifluoroacetic acid in dichloromethane. Fmoc-Lys(Dde)-OH (N-α-Fmoc-N-ε-1-(4,4-dimethyl-2,6-dioxocyclohex-1-ylidene)ethyl-L-lysine) or Fmoc-Lys(ivDde)-OH (N-α-Fmoc-N-ε-1-(4,4-dimethyl-2,6-dioxocyclohex-1-ylidene)-3-methylbutyl-L-lysine) can also be used in this context, wherein the Dde side-chain protecting groups is selectively removed during peptide synthesis by treatment with hydrazine.

For peptide syntheses using Boc chemistry, Boc-Lys(Fmoc)-OH can be used. The side-chain protecting group Fmoc can be selectively removed by treatment with piperidine or DBU (1,8-Diazabicyclo[5.4.0]undec-7-ene) but remains in place when the Boc group is removed from the alpha terminus using trifluoroacetic acid.

As indicated above, in certain embodiments, the linker is an amino acid or other tri-functional moiety positioned between the T_{H} epitope and target epitope.

In related embodiments, the linker is an acidic or basic amino acid. The lipopeptides of the present invention have the lipid moiety attached to a reactive site on the basic or an acidic amino acid. Basic amino acids have at least two amino groups, such as lysine, ornithine, diaminopropionic acid or diaminobutyric acid. Acidic amino acids have at least two carboxy groups and include aspartic acid or glutamic acid.

Attachment of the lipid moiety can be via the alpha amino group or the terminal amino group of the side-chain of the amino acid residue positioned between the T_{H} epitope and target epitope.

Attachment of the lipid moiety can be via the carboxy group of the amino acid or the terminal carboxy group of the side-chain of the amino acid residue positioned between the T_{H} epitope and target epitope.

The target epitope is from a drug of dependence. Drugs of dependence particularly relevant to the present invention are lipophilic drugs, and include, without being limited to, amphetamine, methamphetamine, methylendio, MA, MDMA, cocaine, Δ⁹-tetrahydrocannabinol (and other cannabinoids), morphine (and other opioids of addiction), nicotine and their derivatives. Also contemplated are di- or oligovalent drug target epitopes.

The target epitope is capable of eliciting the production of antibodies when administered to a mammal when part of the lipopeptide carrier. The antibodies generated bind to the drug of dependence for which they are specific, thereby preventing the drug from passing through junctions of the blood-brain barrier and into the brain where the drug would otherwise assert its action.

The target epitope of the present invention may also be from a modified form of a drug of dependence. The drug of dependence may need to be modified so as to allow the drug to be incorporated into the lipopeptides of the present invention.

A variety of methods of attachment of drugs of addiction to the lipopeptide are envisaged and include but are not limited to the use of succinimide and other amide forming reactions, thioether, disulphide bond forming reactions.

The present invention provides a method of eliciting an antibody response against a drug of dependence in a subject, the method comprising administering to the subject a lipopeptide comprising a lipid moiety, a T_{H} epitope, a target epitope specific for a drug of dependence or a drug of dependence and a linker moiety, wherein the linker moiety comprises at least a first, second and third reactive site and wherein the lipid moiety is covalently linked to the first reactive site, the T_{H} epitope is covalently linked to the second reactive site and the target epitope is covalently linked to the third reactive site.

In a further aspect, the present invention provides a method for treating an addiction to a drug of dependence, the method comprising administering to a subject a lipopeptide comprising a lipid moiety, a T_{H} epitope, a target epitope specific for a drug of dependence or a drug of dependence and a linear moiety, wherein the linker moiety comprises at least a first, second and third reactive site and wherein the lipid moiety is covalently linked to the first reactive site, the T_{H} epitope is covalently linked to the second reactive site and the target epitope is covalently linked to the third reactive site.

The present invention also contemplates, the use of a lipopeptide comprising a lipid moiety, a T_{H} epitope, a target epitope specific for a drug of dependence or a drug of dependence and a linker moiety, wherein the linker moiety comprises at least a first, second and third reactive site and wherein the lipid moiety is covalently linked to the first reactive site, the T_{H} epitope is covalently linked to the second reactive site and the target epitope is covalently linked to the third reactive site in the manufacture of a medicament in the treatment or prevention of drug dependency.

The target epitope may be regarded, in one embodiment, as a target B cell epitope. In various aspects, the linker is an amino acid or other tri-functional moiety or analog thereof located between the T_{H} epitope and the target epitope. In related aspects, the linker is an acidic or basic amino acid. In a particular embodiment, the linker is lysine or an analog thereof.

The lipopeptides of the present invention differ in essential aspects from known lipopeptide conjugates of antigens in their enhanced solubility and immunogenicity, and their ability to elicit immune responses without the administration of additional adjuvant. Accordingly, a particular utility of the lipopeptides of the present invention is in the fields of antibody production, synthetic vaccine preparation, diagnostic methods employing antibodies and antibody ligands, and immunotherapy for veterinary and human medicine.

More particularly, the lipopeptide of the present invention induces the specific production of a high titer antibody against the target epitope when administered to an animal subject, without any requirement for an adjuvant to achieve a similar antibody titer. This utility is supported by the enhanced maturation of dendritic cells following administration of the subject lipopeptides (i.e. enhanced antigen presentation compared to lipopeptides having N-terminally coupled lipid).

Accordingly, another aspect of the present invention contemplates a method of eliciting the production of antibody against a target epitope for a drug of dependence comprising administering a lipopeptide comprising a target epitope, a T_{H} epitope, a lipid moiety and a linker moiety having at least a first, second and third reactive site, wherein the T_{H} epitope is covalently linked to the first reactive site, the target epitope is covalently linked to the second reactive site and the lipid moiety is covalently linked to the third reactive site, to the subject for a time and under conditions sufficient to elicit the production of antibodies against the target epitope. In a related aspect, the linker is a lysine residues or lysine analog for covalent attachment of each of the T_{H} epitope, the target epitope and the lipid moiety via an epsilon-amino group of the lysine or via a terminal side-chain group of the lysine analog.

In a related aspect, the linker moiety is lysine and the T_{H} epitope is attached to a carboxyl group, the target epitope is attached an α-amino group and the lipid moiety is attached to the ε-amino group.

In a further aspect, the linker moiety is lysine and the target epitope is attached to the carboxyl group, the T_{H} epitope is attached to the α-amino group and the lipid moiety is attached to the ε-amino group.

In another aspect, the linker moiety is lysine and the lipid moiety is attached to the carboxyl group, the T_{H} epitope is attached to the α-amino group and the target epitope is attached to the ε-amino group.

The effective amount of lipopeptide used in the production of antibodies varies upon the nature of the target epitope, the route of administration, the animal used for immunization, and the nature of the antibody sought. All such variables are empirically determined by art-recognized means.

Reference herein to antibody or antibodies includes whole polyclonal and monoclonal antibodies, and parts thereof, either alone or conjugated with other moieties. Antibody parts include Fab and F(ab)₂ fragments and single chain antibodies. The antibodies may be made in vivo in suitable laboratory animals, or, in the case of engineered antibodies (Single Chain Antibodies or SCABS, etc) using recombinant DNA techniques in vitro.

Alternatively, antibodies may be isolated directly from human subjects who have been immunized using the lipopeptides of the present invention.

In accordance with the present invention, the antibodies may be produced for the purposes of passive immunization of a subject, in which case higher titer or neutralizing antibodies that bind to the target epitope are especially useful. In addition, human monoclonal antibodies generated using the lipopeptides of the present invention will also be useful in the treatment of drug overdoses.

In accordance with this aspect of the present invention, the antibodies may be produced for the purposes of immunizing the subject, in which case high titer of neutralizing antibodies that bind to the target epitope is especially desired. Suitable subjects for immunization will, of course, depend upon whether the subject is a human to be treated or an animal in order to obtain antibodies for humanization. Non-human animals contemplated herein include, farm animals (e.g. horses, cattle, sheep, pigs, goats, chickens, ducks, turkeys, and the like), laboratory animals (e.g. rats, mice, guinea pigs, rabbits) and domestic animals (cats, dogs, birds and the like).

In another embodiment, monoclonal antibodies according to the present invention are “humanized” monoclonal antibodies, produced by techniques well-known in the art. That is, mouse complementary determining regions (“CDRs”) are transferred from heavy and light V-chains of the mouse Ig into a human V-domain, followed by the replacement of some human residues in the framework regions of their murine counterparts. “Humanized” monoclonal antibodies in accordance with this invention are especially suitable for use in in vivo diagnostic and therapeutic methods. Humanized antibodies include deimmunized antibodies.

Alternatively, the antibodies may be for monitoring purposes to ascertain if a subject has developed antibodies to the target epitope.

By “high titer” means a sufficiently high titer to be suitable for use in diagnostic or therapeutic applications. As will be known in the art, there is some variation in what might be considered “high titer”. For most applications a titer of at least about 10³-10⁴ is considered. More particularly, the antibody titer is in the range from about 10⁴ to about 10⁵, even more particularly in the range from about 10⁵ to about 10⁶.

To generate antibodies, the lipopeptide is optionally formulated with a pharmaceutically acceptable excipient. Administration may be intranasal, intramuscular, sub-cutaneous, intravenous, intradermal, intraperitoneal, or by other known routes.

The production of polyclonal antibodies may be monitored by sampling blood of the immunized subject at various points following immunization. A second, booster injection, may be given, if required to achieve a desired antibody titer. The process of boosting and titering is repeated until a suitable titer is achieved. When a desired level of immunogenicity is obtained, the immunized subject is bled and the serum isolated and stored, and/or the subject is used to generate monoclonal antibodies (MAbs).

Any immunoassay may be used to monitor antibody production by the lipopeptide formulations. Immunoassays, in their most simple and direct sense, are binding assays. Certain preferred immunoassays are the various types of enzyme linked immunosorbent assays (ELISAs) and radioimmunoassays (RIA) known in the art. Immunohistochemical detection using tissue sections is also particularly useful. However, it will be readily appreciated that detection is not limited to such techniques, and Western blotting, dot blotting, FACS analyses, and the like may also be used.

The self-adjuvanting lipopeptide is conveniently formulated in a pharmaceutically acceptable excipient or diluent, such as, for example, an aqueous solvent, non-aqueous solvent, non-toxic excipient, such as a salt, preservative, buffer and the like. Examples of non-aqueous solvents are propylene glycol, polyethylene glycol, vegetable oil and injectable organic esters such as ethyloleate. Aqueous solvents include water, alcoholic/aqueous solutions, saline solutions, parenteral vehicles such as sodium chloride, Ringer's dextrose, etc. Preservatives include antimicrobial, anti-oxidants, cheating agents and inert gases. The pH and exact concentration of the various components the pharmaceutical composition are adjusted according to routine skills in the art.

The self-adjuvanting lipopeptide or vaccine is administered prophylactically to a subject who is or has been addicted to a drug dependence or to a naïve subject, (i.e. a subject who has not addicted to a drug of dependence).

The self-adjuvanting lipopeptide or derivative or variant or vaccine composition is administered for a time and under conditions sufficient to elicit a humoral response specific for the addictive drug.

Another aspect of the present invention provides a method of providing or enhancing immunity against a drug of addiction in a subject yet to be exposed to the drug of addiction (i.e. a naïve subject) comprising administering to the subject an immunologically active self-adjuvanting lipopeptide of the present invention or a derivative or variant thereof or a vaccine composition comprising the self-adjuvanting immunogenic lipopeptide or derivative or variant for a time and under conditions sufficient to provide a humoral response against future drug exposure.

The carriers can further comprise any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions. The phrase “pharmaceutically-acceptable” refers to molecular entities and compositions that do not produce an allergic or similar untoward reaction when administered to a human.

The present invention is further described with reference to the following non-limiting examples and accompanying figures. The materials and methods section provided below is relevant to the Examples.

### Chemicals and Reagents

N,N′-dimethylformamide (DMF), acetonitrile (ACN) and disodium hydrogen phosphate (Na₂HPO₄) were obtained from Merck (Damstadt, Germany) and dichloromethane (DCM) from Merck, (Victoria, Australia). Trifluoroacetic acid (TFA) and 1,3 diisopropylcarbodiimide (DICI) were obtained from Auspep, (Parkville, Australia). 1-hydroxybenzotriazole (HOBt; CEM) and O-benzotriazole-N,N,N′,N′-tetramethyl-uronium-hexafluorophosphate (HBTU) were purchased from Novabiochem, (Damstadt, Germany). Diethyl ether and hydrogen peroxide (H₂0₂) were obtained from Merck (Kilsyth, Australia) diisopropylethylamine (DIPEA), 1,8-diazabicyclo-[5.4.0]undec-7-ene (DBU), Bovine serum albumin (BSA), dimethylaminopyridine (DMAP), triisopropylsilane (TIPS) and tween-20 were obtained from Sigma-Aldrich, Steinheim, Germany. 2,4-dinitrophenol was obtained from (Calbiochem, San Diego, Calif., USA), palmitic acid from Merck (Hohenbrunn, Germany), phenol from BDH (Laboratory Supplies, Poole, England), and sodium azide and citric acid from (Chem-supply, Gillman, South Australia). Amino acids were purchased from either Auspep (Melbourne, Australia) or Novabiochem (Damstadt, Germany). All chemicals used were of analytical grade or its equivalent unless otherwise stated.

### Target Epitopes

The target epitopes used herein included the hapten 2,4-dinitrophenol (DNP), as a proof of principle example and amphetamine and the stable cocaine derivative, norcocaine, for the substances of abuse studies.

### Synthesis of Vaccine Constructs Targeting DNP

### Synthesis of T Helper Epitopes

In the synthesis of peptide-based vaccine constructs, three T_{H} cell epitopes were used (Table 1).

TABLE 1
Abbreviations and amino acid
sequences of the T helper epitopes

| Abbreviation | Peptide Amino Acid Sequence |
|---|---|
| T_{H(MV)} | KLIPNASLIENCTKAEL |
| T_{H(flu)} | ALNNRFQIKGVELKS |
| T_{H(ova)} | ISQAVHAAHAEINEAGR |

All T_{H} epitopes used in the design of completely synthetic peptide vaccines in this study were synthesized automatically using a CEM Microwave Peptide synthesizer on Tentage) S-RAM resin (RAPP Polymere, Tubingen substitution factor=0.23 nmol/g) in the solid-phase using fluorenylmethoxycarbonyl (Fmoc) chemistry. The T_{H} epitope T_{H(MV)} is derived from the fusion protein of the canine distemper virus, morbillivirus, and known to be a potent stimulator of helper T cells in many animals including mice. The T_{H} epitope T_{H(Flu)} was derived from the a-chain of haemagglutinin of Mem71 influenza virus and is active in BALB/c mice and T_{H(ova)} was derived from chicken ovalbumin, shown to be active in C57/B6 mice.

Incorporating the Target Epitope DNP into Peptide Constructs

An Fmoc-lysine-(mtt) residue was manually coupled to the N-terminus of the T_{H} epitope synthesized by CEM peptide synthesizer, while still attached to the resin. After Fmoc-α-amino deprotection 2,4-dinitrophenol-ε-amino-n-caproic acid (2,4-DNP-ahx; Sigma, Steinheim, Germany) was coupled to the amino group of the N-terminal lysine residue and through the carboxyl group present on the caproic acid (FIG. 1). Cleavage of the peptide from resin yielded the non-lipidated version of the DNP-peptide construct (DNP-T_{H}). 2,4,6-trinitrobenzenesulfonic acid (TNBSA; Fluka, Buchs, Switzerland) tests confirmed the completion of reaction manual synthesis.

The lipidated version of the DNP-peptide constructs were synthesized by the attachment of S-(2,3-bis[palmitoyloxy]propyl)cysteine (P2C) in a branched configuration through the ε-amino group of a lysine residue positioned between the target and T_{H} epitopes. The Mtt protective group on the ε-amino group of the central lysine was removed by washing the resin in 1% TFA w/v in DCM every five minutes for an hour. Two serine residues were then coupled to the free ε-amino group in tandem. A four-fold excess of Nα-Fmoc-S-(2,3-dihydroxypropyl)-cysteine(Fmoc-Cys(Dhp)) was coupled to the terminal serine in the presence of HOBt, and DICI dissolved in DMF. Finally, two palmitic acid molecules were coupled to the Fmoc-Cys(Dhp) using a 20 fold excess of palmitic acid, a 2 fold excess of DMAP, and a 20 fold excess of DICI dissolved in DCM. The reaction was held at room temperature for 16 hours after which the Fmoc group present on the Cys(Dhp) was removed and the resin dried under vacuum after washing in ACN.

Divalent DNP constructs used in this study were synthesized by coupling Fmoc-lysine-(Fmoc)-OH (Auspep, Australia) to the N′-terminus of the peptide before the attachment of the target epitopes. Fmoc deprotection of the peptide exposed two potential binding sites to which DNP-ahx was conjugated to the peptide by acylation for 60 minutes, yielding a divalent construct.

Cleavage of Peptide from the Solid-Phase Support

Peptide was cleaved from the resin along with side chain protecting groups of the amino acids by mixing for two hours in Reagent B (88% v/v TFA, 5% v/v phenol, 5% v/v ddH₂O, and 2% v/v TIPS). The peptides were separated from the resin by filtration, before concentration under a stream of nitrogen gas. Precipitation of the peptide was achieved by suspension and centrifugation in −18° C. diethyl ether twice. The final supernatant was discarded and the sedimented peptide dissolved in 50% ACN in ddH₂O before lyophilization.

### Peptide and Lipopeptide Purification and Analysis

Reverse phase high performance liquid chromatography (RP-HPLC) using a VYDAC protein C4 column (10 mm×250 mm; Alltech, NSW, Australia) installed in either a Waters Semi-Preparative (Millipore, Milford, Mass., USA) or a GBC (GBC Scientific Equipment, Australia) HPLC system were used for the purification of all peptides and lipopeptides used in this study. Immunogens were isolated from impurities at a flow rate of 2.5 ml/min using the following linear gradients for all non-lipidated peptides: 5 min 0% B, 30 min 40% B, 31 min 100% B, 32 min 0% B, where A=H₂O with 0.1% TFA and B=ACN with 0.1% TFA. All lipidated peptides were purified using the following gradient: 5 min 0% B, 46 min 82% B, 47 min 100% B, 48 min 0% B. Collected fractions were analysed for purity with analytical RP-HPLC on a Waters 996 HPLC system, using a VYDAC C4 column (10 mm×250 mm).

Electrospray Ionisation Mass Spectrometry (ESI-MS) on an Agilent 1100 series LC/MSD Trap system (Agilent Technologies, Waldbroom, Germany) set to a positive polarity mode was utilized for mass analysis of each peptide and lipopeptide. Deconvolution of the charge series was achieved using Bruker Data Analysis 2.1 software (Agilent Technologies).

Synthesis of DNP-BSA

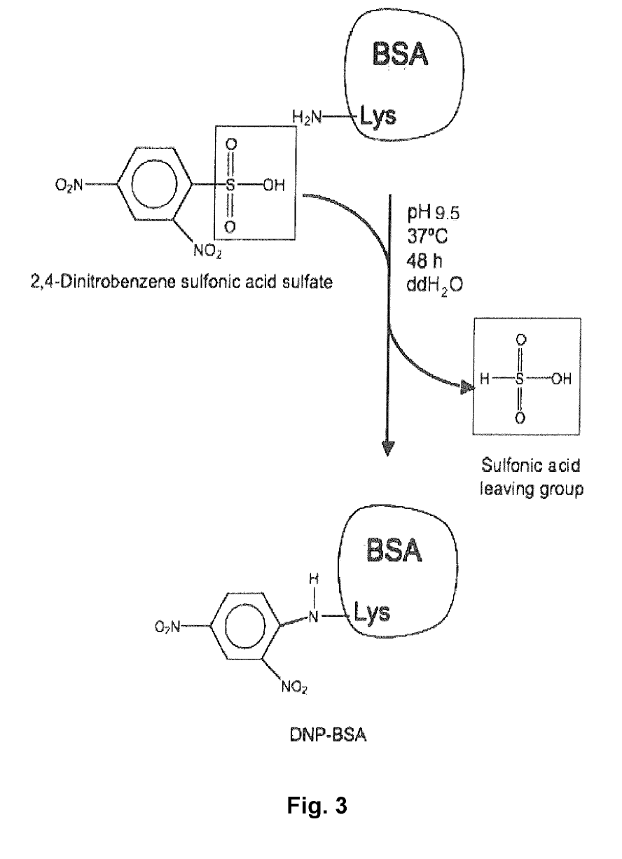

Using a modified method from Yokoyama et al. Molecular Immunology 29(7-8):935-947, 1992, 30 mg 2,4-dinitrobenzene sulfonic acid (Aldrich, Steinheim, Germany) was reacted with 5 mg BSA in ddH₂O adjusted to pH 9.5 with potassium carbonate at 37° for 48 hours in the absence of light (FIG. 3). The protein-DNP conjugate was purified by size exclusion chromatography on a Superdex™ 75 column (10 mm×300 mm) installed in a Pharmacia fast performance liquid chromatography (FPLC) system. The running solvent was PBS at a flow rate of 0.5 ml/min.

Determining Substitution of DNP-BSA

The absorbance of DNP-BSA was determined by UV spectrophotometry at 360 nm. This enabled the calculation of the substitution rate of DNP coupling to BSA taking into account the recovery rate of BSA in PBS and the molar extinction coefficient of DNP (17,530 M⁻¹cm⁻¹). UV spectrophotometry was also utilized to determine the percentage purity of DNP-peptide and lipopeptide vaccines following the construction of a standard curve for DNP, which allowed for more accurate calculation of amount used for immunizations.

### Synthesis of Peptide-Based Vaccines Targeting Amphetamine, Cocaine and Morphine

### Modification of Amphetamine to Facilitate its Attachment to Peptide

D-amphetamine sulfate was succinylated by the addition of a 2-fold molar excess of succinic anhydride (Aldrich, Steinheim, Germany) in the presence of a 3-fold molar excess of triethylamine (Ajax Chemicals, Sydney, Australia) dissolved in ethanol (Chemsupply, Gillman, South Australia). This mixture was reacted for 16 hours at 37° C. Solvent was then evaporated by the application of a vacuum at 60° C. using the Rotavapor apparatus (Buchi, Switzerland). Succinylated amphetamine was stored at 4° C. until required.

### Coupling of Succinylated Amphetamine to Peptide

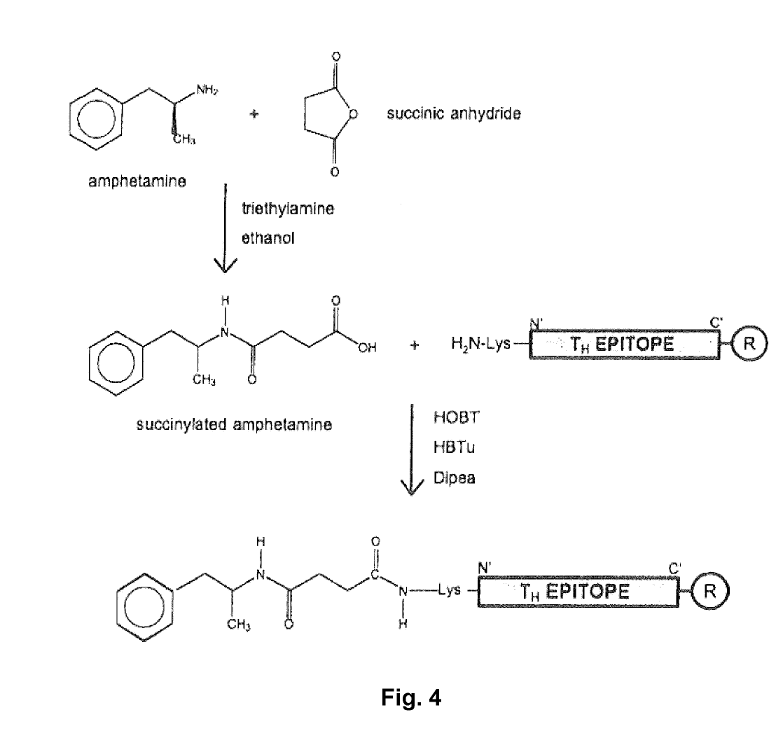

Succinyl amphetamine was coupled to the N′ terminal lysine residue of peptide constructs through the carboxyl group in a 60 minute acylation reaction in the presence of the same amounts of the same activators used for amino acid addition (HOBt, HBTU and DIPEA) (FIG. 4).

### Coupling of Cocaine Derivative to Peptide

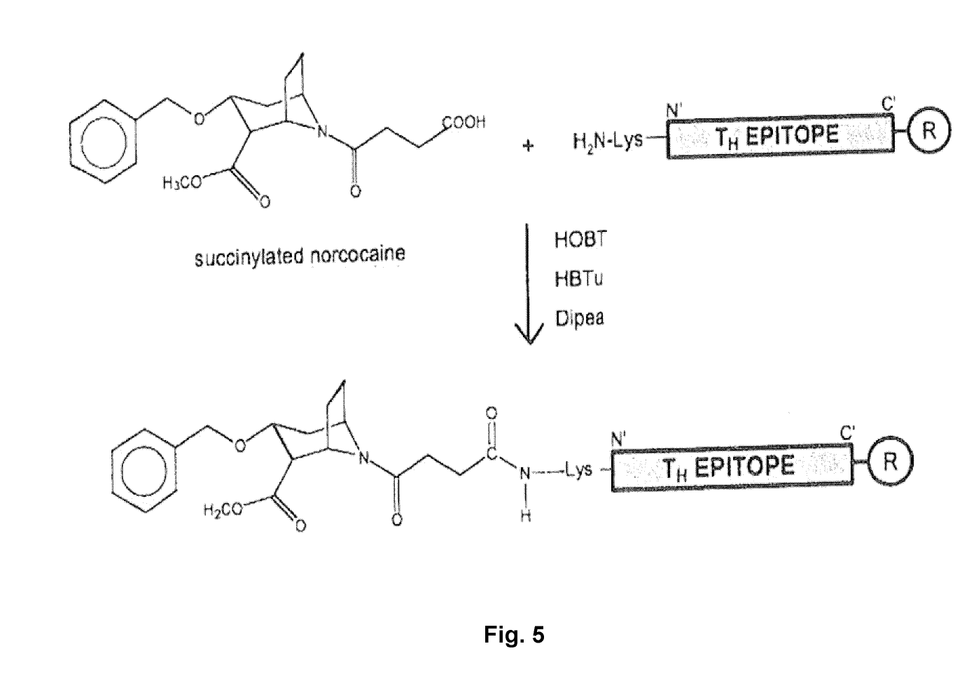

Succinyl norcocaine was conjugated to peptide constructs in the same way as succinyl amphetamine (FIG. 5).

Incorporating the Target Epitope Morphine into Peptide Constructs

Morphine was modified to generate 6-succinyl-morphine following similar procedures as described above for the modification of amphetamine

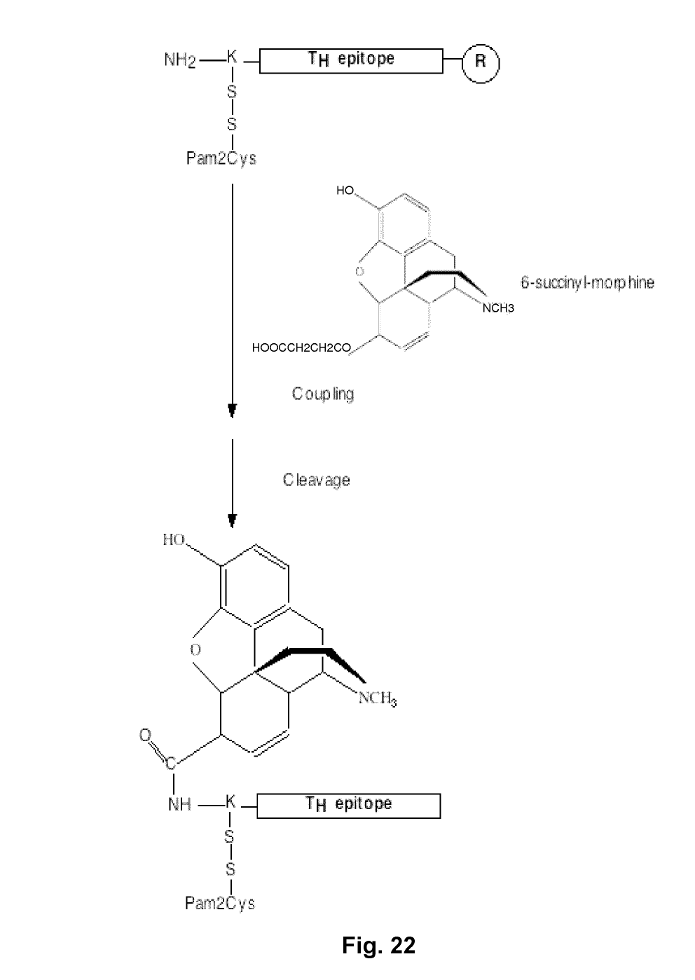

To Incorporate the target epitope morphine into peptide construct a Dde-lysine-(Fmoc) residue (Merck Australia) was manually coupled to the N-terminus of the T_{H} epitope (T_{H(ova)} synthesized by CEM peptide synthesizer, while still attached to the resin. After Fmoc-ε-amino deprotection, two serine residues were then coupled to the free ε-amino group in tandem. A four-fold excess of Fmoc-Cys(Dhp) was coupled to the terminal serine in the presence of HOBt, and DICI dissolved in DMF. Finally, two palmitic acid molecules were coupled to the Fmoc-Cys(Dhp) using a 20 fold excess of palmitic acid, a 2 fold excess of DMAP, and a 20 fold excess of DICI dissolved in DCM. The reaction was held at room temperature for 16 hours after which the Fmoc group present on the Cys(Dhp) was removed and the exposed amino group was blocked with tert-butyloxycarbonyl (BOC) group. The peptide resin was treated two time of five minutes with 2% hydrazine in DMF to remove Dde protecting group on the lysine residue and 6-succinyl morphine was coupled to the exposed α-amino group using similar conditions as in the coupling succinylated amphetamine to peptide. See FIG. 22 for diagrammatic representation of construct.

### Conjugation of Amphetamine to BSA

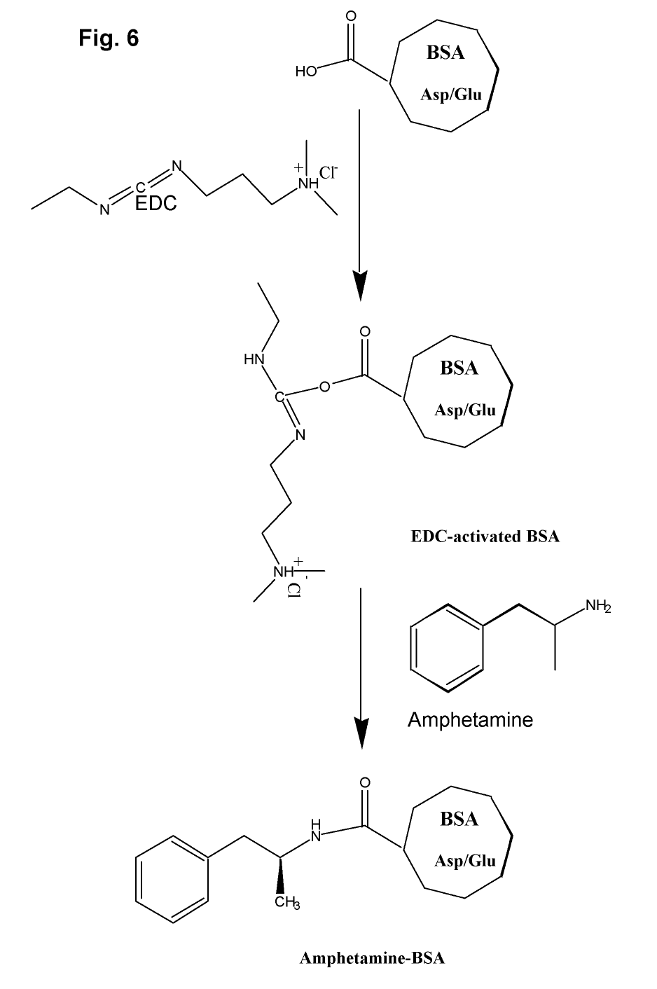

Amphetamine was conjugated to BSA using carbodiimide chemistry. Briefly, 100 mg D-amphetamine sulfate (Sigma) was added to 25 mg BSA dissolved in 2.5 mL ddH2O. N-(3-dimethylaminopropyl)-N′-ethyl-carbodiimide hydrochloride (EDC; Fluka, Steinheim, Germany) was added to the mixture and stirred for 1 hour at room temperature (FIG. 6). The reaction mixture was then extensively dialysed for 24 hours at room temperature against saline using Selbys dialysis membrane (molecular weight cut off at 12,000; Union Carbide Corporation, Illinois, USA) to remove free-hapten. The substitution rate of amphetamine to BSA was then determined by UV spectrophotometry.

### Conjugation of Cocaine-Derivative to BSA

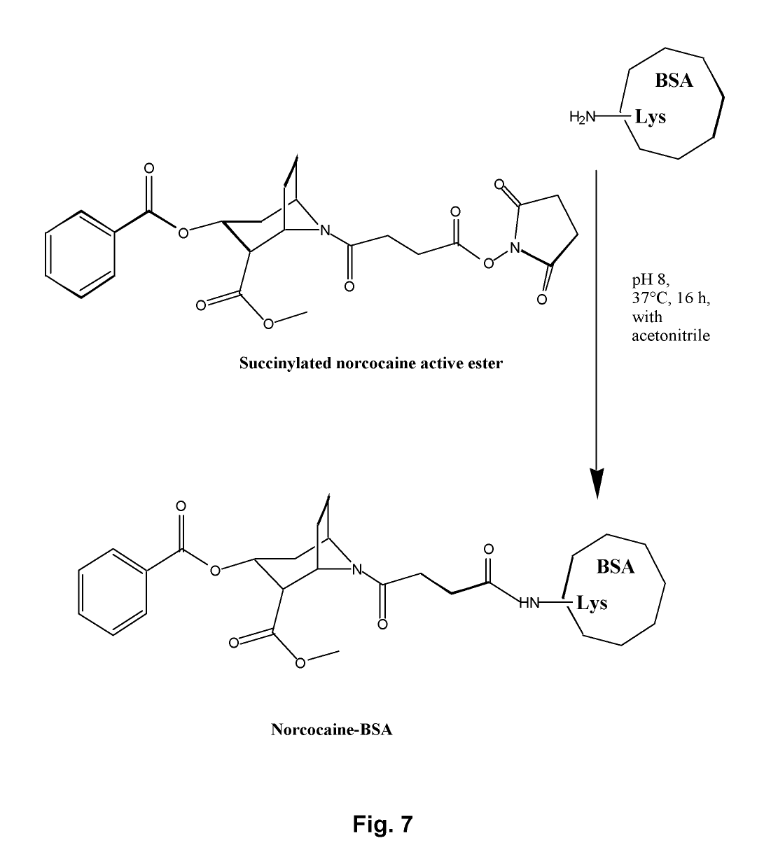

30 mg of an active ester derivative of succinyl norcocaine was dissolved in a small volume of acetonitrile before adding to 6 mg BSA dissolved in PBS. The reaction mixture was adjusted to pH 8 and incubated for 16 hours at 37° C. yielding an insoluble product (FIG. 7). Free hapten was removed by dialysis against saline at room temperature for 24 hours and the product was stored at 4° C. prior to use.

### Immunization Protocol

6 to 10 week old BALB/c and C57/B6 mice were bred and maintained in an experimental animal facility. Mice were each immunized subcutaneously with 20 nmol of peptide in 100 μL sterile saline at base of tail. For carrier-protein vaccine immunisation, mice were administered with 100 μg of hapten-BSA.

Non-lipidated peptides and protein-carrier based vaccines were emulsified in complete Freunds's adjuvant for the primary and incomplete Freund's adjuvant (CFA and WA respectively; Sigma-Aldrich) for the secondary inoculation for subcutaneous administration. Groups of five mice were primed on day 0, and again on day 21 with the same lipopeptide construct(s), using the same route of inoculation. Mock immunization with sterile saline and non-adjuvanted peptides and proteins were used as negative controls for each study.

### Collection and Preparation of Antibody

Mice were bled on day 21 and again on day 31 for primary and secondary sera, respectively.

### Intranasal Immunisation

Along with subcutaneous (s.c.) immunisation as per DNP-study, an intranasal (i.n) immunisation schedule for amphetamine vaccine candidates was also undertaken. Mice were anaesthetised with Penthrane before administration of 20 nmol of peptide immunogen in 50 μl of saline on day 0 and day 21. Sera were collected on day 28 to assess total and IgA antibody production and saliva on day 29 to assess the production of secretory IgA antibody. Saliva was extracted after Penthrane anaesthesia and induction by carbamoylcholine chloride (Sigma-Aldrich). The saliva was stored at −20° C. before usage 24 hours later.

### Detection of Anti-DNP Antibody by Enzyme Linked Immunosorbent Assay (ELISA)

Direct-binding ELISAs were performed in 96-well polyvinyl flat-bottomed microtiter plates (Pathtech, Heidelberg West, Victoria, Australia) with all incubations carried out at room temperature (RT) in a humidified atmosphere. Plates were coated overnight with 50 μl/well of antigen at a concentration of 1 μg/ml protein or 51.×g/m1 peptide in phosphate-buffered saline (PBS; 0.15M NaCl, 20 mM Na₂HPO₄, pH 7.4) containing 0.1% sodium azide (Chem-supply, Gillman, South Australia) (PBSN₃). Unbound antigen was discarded and wells blocked for 2 hours with 100 μl of 10% v/v bovine serum albumin in PBS (BSA₁₀PBS) per well. Blocking solution was removed by washing 4 times with PBS containing 0.05% v/v Tween-20 (PBST; Tween-20, Aldrich, Steinheim, Germany). Half-log serial dilutions of sera in 50 μl BSA₅PBST (PBST containing 5% v/v BSA) were prepared across the plate. Sera were allowed to bind overnight before discarding and washing wells six times with PBST. 50 μl of a 1/400 dilution of horseradish peroxidase-conjugated rabbit anti-mouse immunoglobulin (HRP-RαM; Dako, Denmark) in BSA₅PBST, was added to each well and incubated for 2 hours. The conjugate was then discarded and wells washed 6 times with PBST. 100 μl of substrate (50 mM citric acid containing 1/200 dilution of ABTS and 1/250 dilution of H₂0₂ at pH 4 was added to each well and plates were incubated for 12-15 minutes to allow adequate colour development. To stop the reaction, 50 μl of 50 mM sodium fluoride was added to each well. Optical densities (O.D.) were measured using a Multiskan plate reader (Labsystems, Finland) at a dual wavelength of 405 nm and 450 nm. Antibody titres were determined as the reciprocal logarithmic dilution of the test sera which gave an absorbance reading of 0.2 or five times the O.D observed for background (irrelevant sera and no-antigen coat controls). An arbitrary value of 1 was assigned to tires of less than 2 for generating graphs.

### Inhibition ELISA

Competitive ELISA assays involved an extra step whereby ½ log dilutions of inhibitors in BSA5PBST beginning at 25 nmol/well were added to a constant limiting concentration of antibody in BSA₅PBST and incubated for 2 hours on separate polyvinyl plates during the blocking step of a regular ELISA. 50 μl/well of the inhibitor and sera mixtures were then transferred to coated, blocked and washed plates and incubated overnight. Plates were then conjugated with HRP-RαM, developed, and read as per ELISA. The percentage of inhibition was determined by the following formula:

$$\% inhibition = ( 1 - \frac{\begin{matrix}Absorbance ( with inhibitor ) - \\ background\end{matrix}}{\begin{matrix}Absorbance ( without inhibitor ) - \\ background\end{matrix}} ) \times 100 \%$$

### IgA Isotyping ELISA

An ELISA was conducted to detect IgA antibodies specific for amphetamine elicited by intranasal immunisation using the sera as well as saliva samples. For isotyping ELISAs, Goat anti mouse IgA-HRP (Southern Biotech Birmingham, Ala., USA) were used as secondary antibodies, replacing HRP-RαM in a direct binding ELISA.

### Statistical Analysis

All P-values were calculated using a one-way ANOVA with a 95% confidence interval using the Tukey test algorithm for post-test analyses.

### Example 1

### Synthesis and Immunological Study of Peptide-Based DNP Vaccines

2,4-Dinitrophenol (DNP) has been studied extensively for its properties as a hapten. When conjugated to a carrier protein and administered in adjuvant to animals, anti-DNP antibodies are induced. To show that a completely synthetic self-adjuvanting lipopeptide construct also has the ability to elicit anti-hapten antibody production, DNP was conjugated to completely synthetic self-adjuvanting lipopeptide constructs as well as a protein-carrier and administered to animals.

### Synthesis of DNP-BSA

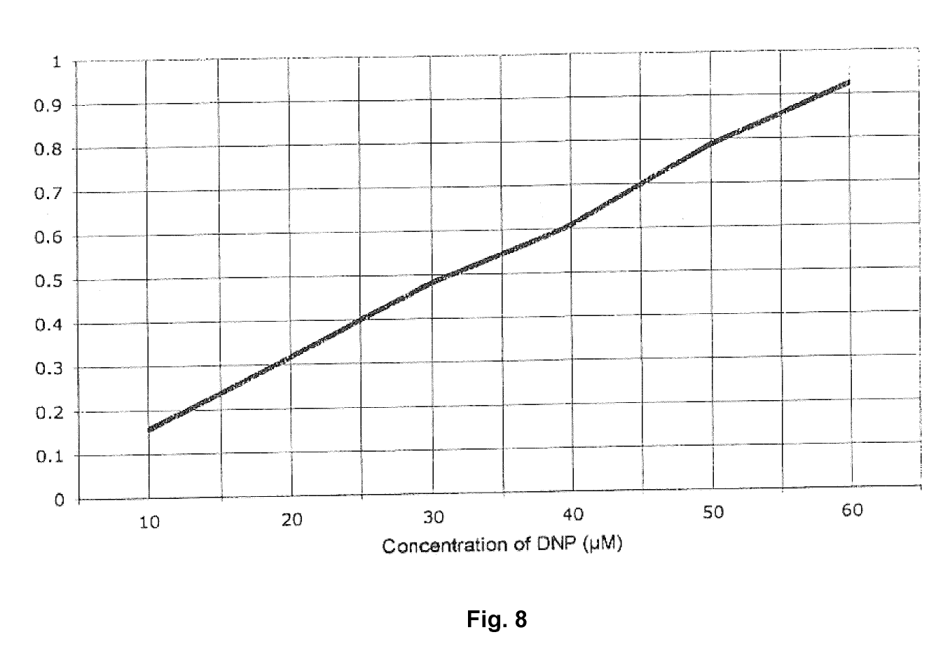

2,4-DNP was successfully conjugated to BSA following a modified method by Yokoyama et al. 1992 supra. Briefly, 2,4-DNP was attached to BSA through the C-amino group present on the side chain of lysine residues (FIG. 3), of which BSA has 60. The recovery rate of BSA after purification in PBS by FPLC was 84%. An optical density standard curve (produced by increasing concentrations of 2,4-DNP-glycine; molar extinction coefficient; 17,530 M⁻¹cm⁻¹ at λ=360 nm; (FIG. 8) was used to determine the substitution rate of 2,4-DNP onto lysine residues of BSA. It was found that under optimal reaction conditions, a substitution rate of between 18-22 2,4-DNP molecules per BSA was achieved.

### Preparation of Completely Synthetic Peptide-Based DNP-Vaccines

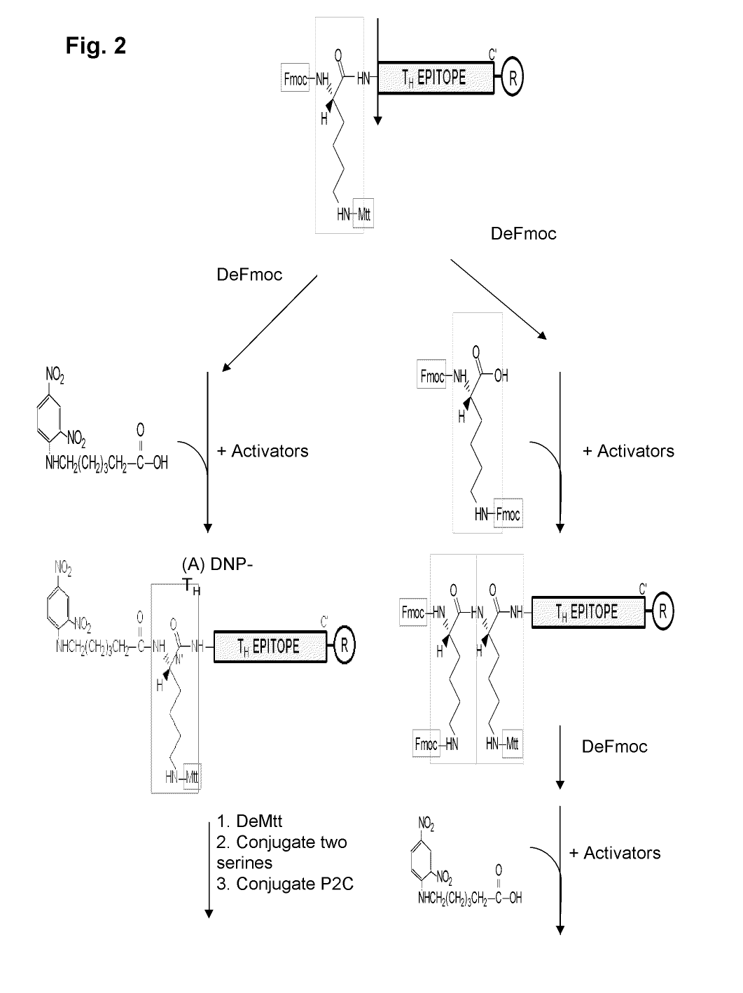

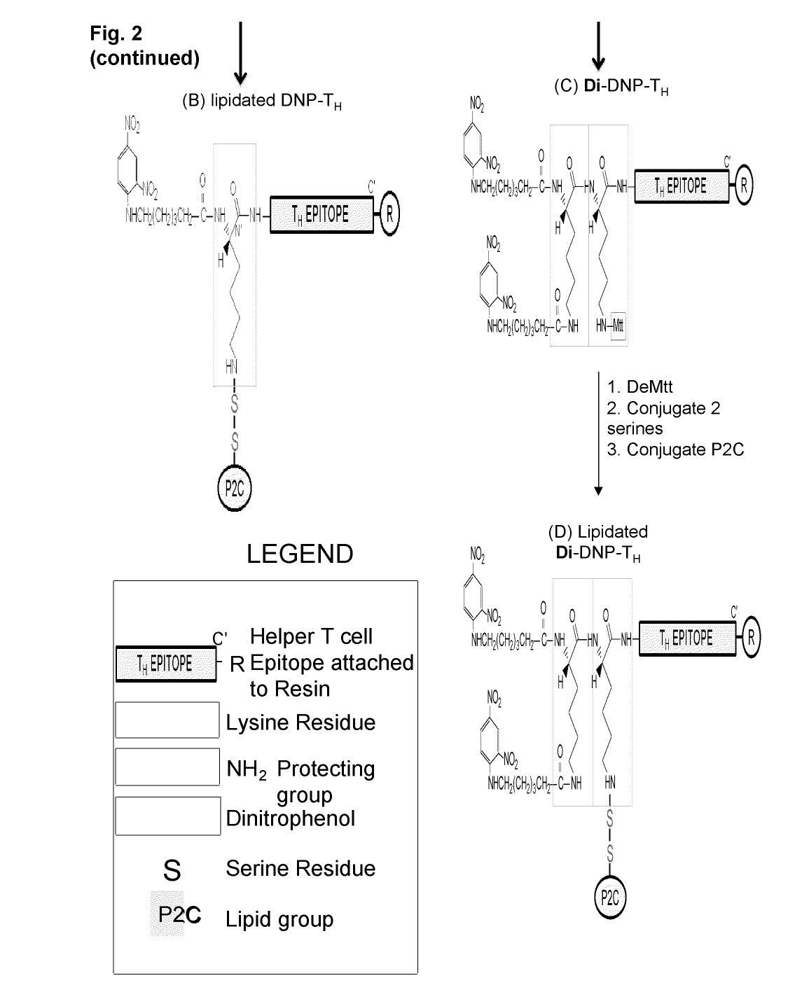

All synthetic DNP peptide-based constructs were synthesized following the schematic outlined in FIG. 2.

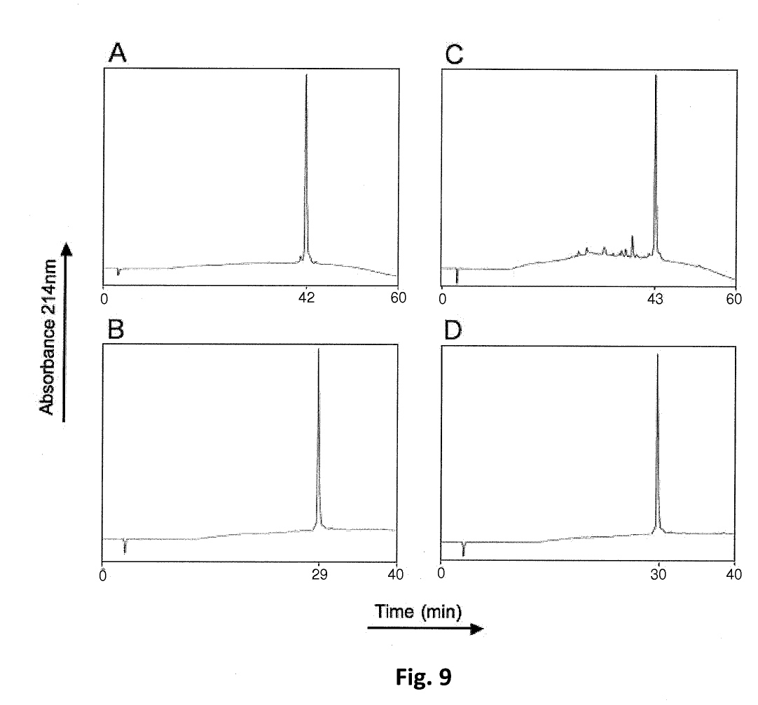

Analytical chromatograms and results of mass spectrometric characterisation of purified peptide constructs used to immunize mice are shown in FIG. 9, and Table 2, respectively. Purified and lyophilized peptide-based DNP vaccines were dissolved in saline and assessed by UV spectrophotometry to guarantee the correct dose of DNP administered to mice.

TABLE 2
Theoretical and actual mass in Daltons of DNP-TH
constructs as determined by ESI-MS

| | Theoretical | Observed |
|---|---|---|
| Construct | mass | mass |
| Lipidated DNP-T_{H(MV)} | 3,091.9 | 3,091.73 |
| Lipidated DNP-T_{H(Flu)} | 3,479.9 | 3,079.33 |
| Lipidated DNP-T_{H(ova)} | 3,136.1 | 3,136.06 |
| Non lipidated DNP-T_{H(Flu)} | 2,251.7 | 2,250.90 |
| Non-lipidated DNP-T_{H(ova)} | 2,307.9 | 2,308.39 |
| Lipidated DiDNP-T_{H(Flu)} | 2,659.2 | 2,659.06 |
| Non Lipidated DiDNP-T_{H(Flu)} | 2,251.7 | 2,250.90 |
| Lipidated DiDNP-T_{H(ova)} | 3,543.6 | 3,543.84 |
| Non-lipidated DiDNP-T_{H(ova)} | 2,715.4 | 2,715.84 |

### Evaluation of the Ability of Peptide-Based DNP-Vaccines to Elicit DNP-Specific Antibody

The vaccination schedule involved primary inoculation at day 0, bleeding for primary sera and boosting with the same dose on day 21 and bleeding again on day 31 for secondary sera.

### Detection of Anti-DNP Antibody in BALB/c

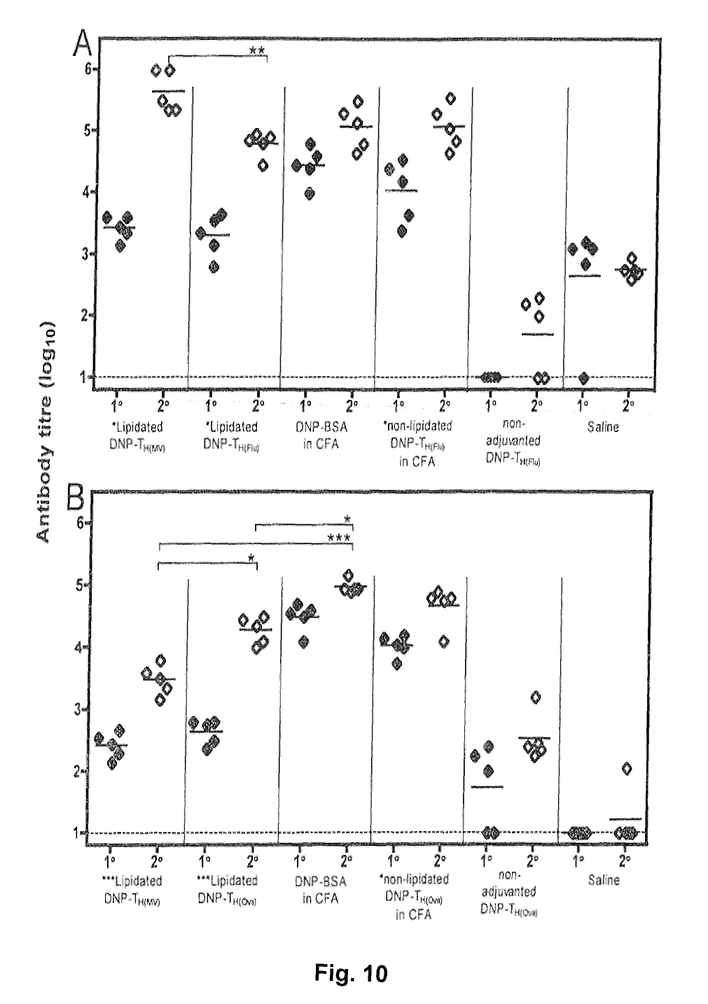

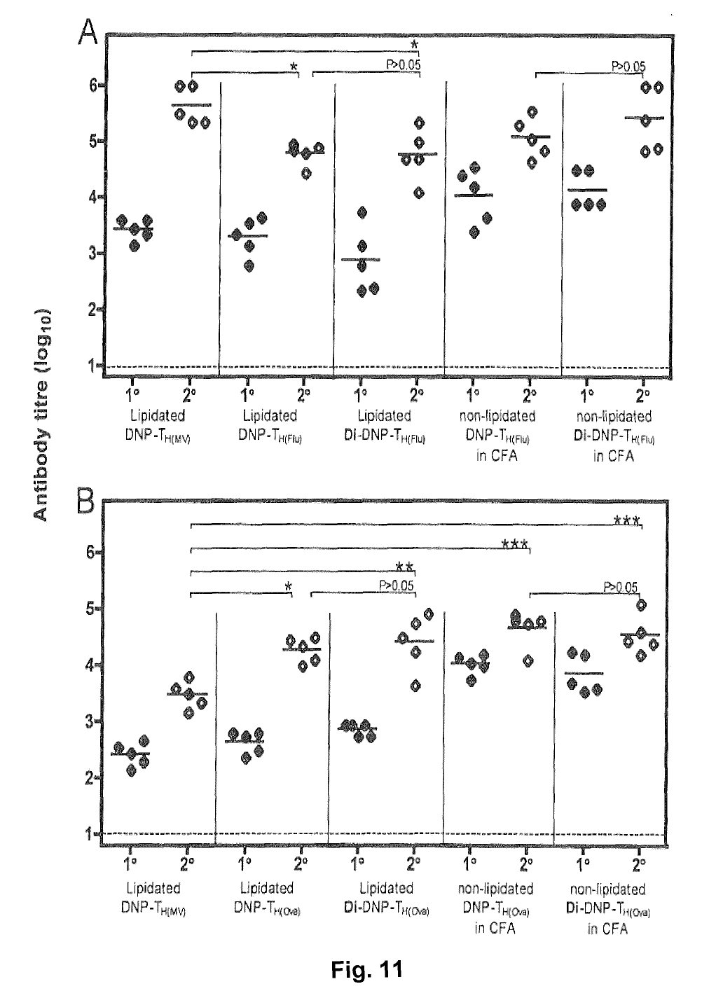

Two lipidated DNP-T_{H} constructs were tested in BALB/c mice (FIG. 10, panel A). These constructs contained one of two T_{H} epitopes (T_{H(Mv)} and T_{H(FIu)}), both which have been previously shown to be active in this strain. Although the primary responses elicited by both lipidated DNP-T_{H} constructs were similar to the saline control, after secondary boost, both groups demonstrated a significant increase (P<0.05) in the secondary antibody titre detected by ELISA. The antibody titre of the secondary sera induced by lipidated constructs and non-lipidated DNP-T_{H(Flu)} construct administered in CFA were similar in magnitude (P>0.05) to the secondary sera from mice administered with the DNP-BSA in CFA positive control. Furthermore, it was observed that in BALB/c mice, the secondary antibody titre of approximately 10^{5.5} elicited by DNP-T_{H(MV)} was statistically higher (P<0.01) than that of the secondary response elicited by DNP-T_{H(Flu)}, indicating T_{H(MV)} a stronger helper T cell epitope compared to T_{H(Flu)} in BALB/c mice.

### Detection of Anti-DNP Antibody in C57/B6

FIG. 10, panel B shows that when administered to C571B6 mice, lipidated DNP-T_{H(MV)} and DNP-T_{H(ova)} were able to induce primary and secondary antibody responses significantly greater than the non-adjuvanted DNP-T_{H(Ova)} and saline controls (P<0.001). Also, the secondary responses elicited by the lipidated DNP-T_{H} constructs yielded a statistically significant increase on the primary responses (P<0.001). Unlike in BALB/c mice, although the lipidated construct incorporating the T_{H} epitope derived from morbillivirus elicited a statistically significant antibody response, it was relatively low compared to the DNP-BSA administered in CFA positive control and was not as immunogenic as the lipidated constructed incorporated with the T_{H} epitope derived from chicken ovalbumin. Lipidated DNP-T_{H(ova)} administered mice recorded statistically higher anti-DNP antibody titres in the secondary response (P<0.05) compared to the secondary sera of lipidated DNP-T_{H(MV)}. The group of mice inoculated with the non-lipidated DNP-T_{H(ova)} administered in CFA displayed primary and secondary antibody responses similar in magnitude to that of mice inoculated with the DNP-BSA administered in CFA positive control.

Overall, the self-adjuvanting lipidated hapten-T_{H} constructs were able to induce high levels of antibody in the secondary response. Additionally, the non-lipidated peptides, DNP-T_{H(Flu)} and DNP-T_{H(ova)} administered in CFA to BALB/c and C57/B6 mice respectively, were able to induce primary and secondary antibody responses as high as the DNP-BSA administered in CFA positive control. This study also demonstrated that the T_{H} epitopes incorporated into vaccines has an effect on the size of antibody response elicited in different strains of mice.

### Example 2

### Divalent DNP-Peptide Immunogen Study

It was investigated whether a divalent DNP-peptide construct could induce an enhanced immune response compared to constructs coupled with a single copy of DNP (FIG. 11).

Sera from BALB/c mice which were inoculated with lipidated DiDNP-T_{H(Flu)}, or lipidated DNP-T_{H(Flu)} in saline and non-lipidated DiDNP-T_{H(Flu)} in CFA were tested for anti-DNP antibody titre. The results show no significant difference in antibody titres amongst these groups (FIG. 11, panel A). Similarly, when C57/B6 mice were inoculated with either the lipidated DiDNP-T_{H(OVA)}, or lipidated DNP-T_{H(OVA)} administered in saline or non-lipidated DiDNP-T_{H(OVA)} administered in CFA, the antibody titres (FIG. 11, panel B) obtained from the sera of these groups of mice were statistically indistinguishable.

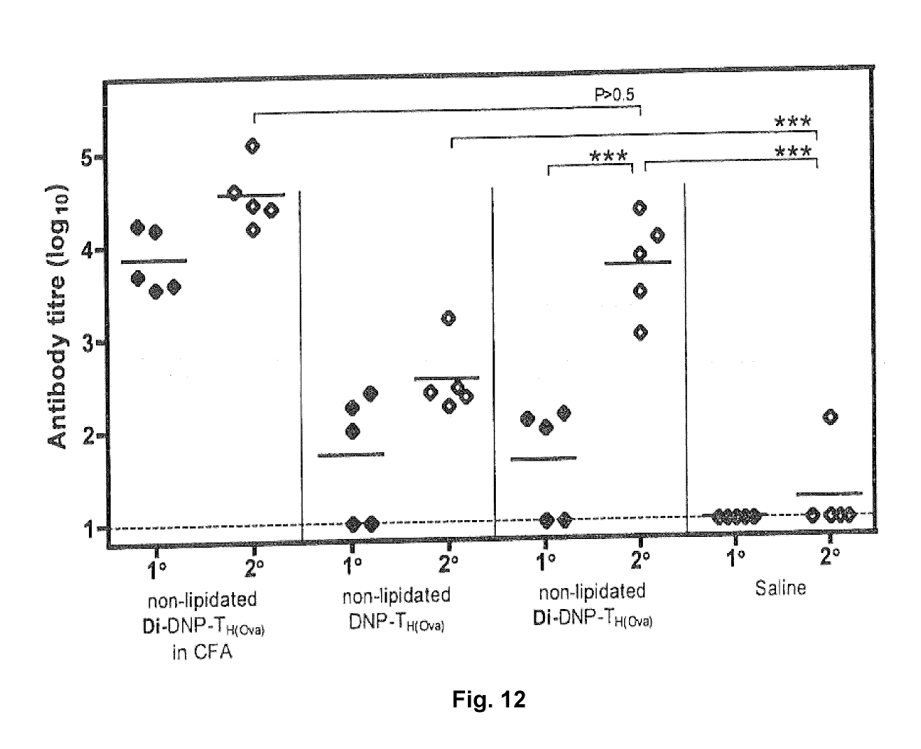

Interestingly, although lipidated divalent peptide-based DNP vaccines did not appear to be more immunogenic, in C57/B6 mice, it was observed that the non-adjuvanted divalent DNP-T_{H(ova)} control could elicit significant primary and secondary anti-DNP antibody responses. The secondary antibody titres from mice administered non-lipidated Di-DNP-T_{H(ova)} and mice inoculated with Di-DNP-T_{H(ova)} administered in CFA were statistically indistinguishable (FIG. 12). This indicates that a divalent DNP-T_{H(ova)} construct is sufficiently immunogenic in C57/B6 mice, thus adjuvanting the immunogen may not be necessary to stimulate an antibody response. (Non-lipidated Di-DNP-T_{H(Flu)} was not tested in mice due to limited animal supplies).

### Example 3

### A Strategy to Overcome MHC Class Restriction in Peptide-Based Vaccines by Using Constructs Incorporating Promiscuous T_{H} Epitopes or Mixtures of Peptide-Based Constructs Containing T_{H} Epitopes Active in Different Strains

A limitation of using peptide-based vaccines is that the incorporated T_{H} epitope is normally only active in one species or strain of animal due to MHC class restriction. To overcome this limitation, either a promiscuous T_{H} epitope (such as T_{H(Mv)}) or a mixture of two or more peptide vaccines needs to be used to broaden the coverage of MHC types. T_{H(Flu)} has been reported to be active in BALB/c and T_{H(ova)} in C57/B6 mice. A vaccine combining the lipidated DNP-T_{H(ova)} and lipidated DNP-T_{H(Flu)} was tested to determine whether anti-DNP antibody responses could be induced in both strains of mice.

An amount of 20 nmol of lipidated DNP-T_{H(Ova)} and lipidated DNP-T_{H(Flu)} were administered separately into mice and the ensuing antibody responses compared to that obtained from mice receiving a 20 nmol dose of a mixture containing 10 nmol of each. DNP-BSA administered in CFA provided the positive control. Mock immunization with saline gave antibody titres less than or similar to the secondary sera from mice given the non-adjuvanted mixture (antibody titre <10² in both BALB/c and C57/B6 mice).

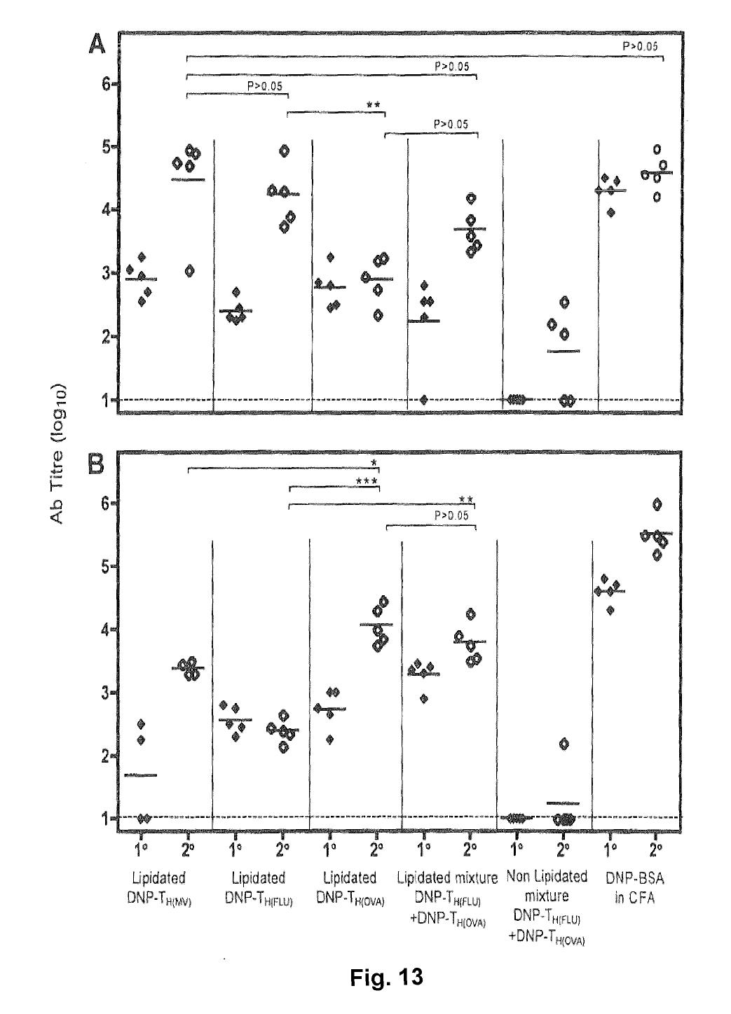

Lipidated DNP-T_{H(MV)} when administered to BALB/c mice was able again to elicit a secondary antibody response similar in magnitude to that of the DNP-BSA administered in CFA positive control and the lipidated DNP-T_{H(Flu)} (FIG. 13, panel A). A statistically significant, but low (significantly less than that of lipidated DNP-T_{H(Flu)} and lipidated DNP-T_{H(MV)}) secondary antibody titre was detected for lipidated DNP-T_{H(ova)}. However, when a mixture of the lipidated DNP-T_{H(ova)} and lipidated DNP-T_{H(Flu)} in equal parts were administered to mice, the secondary antibody responses detected were not significantly different to the highest antibody responses, induced by lipidated DNP-T_{H(MV)} (P>0.05) and the DNP-BSA administered in CFA positive control.

On the other hand, although lipidated DNP-T_{H(MV)} was able to elicit a significant secondary response (P<0.001) in C57/B6 mice, the response was not as high as that of DNP-T_{H(ova)} (FIG. 13, panel B). As expected, lipdated DNP-T_{H(ova)} induced a significantly higher (P<0.0001) secondary antibody response than lipidated DNP-T_{H(Flu)}. When a mixture of lipidated DNP-T_{H(Flu)} and lipdated DNP-T_{H(ova)} was used, the antibody titre observed was strong and statistically indistinguishable from that of sera of mice administered the lipidated DNP T_{H(Ova)}.

Overall, it was found that T_{H(MV)} is a promiscuous T_{H} epitope as it was able to elicit significant secondary antibody responses in both strains of mice. However, although it has been shown to be the best inducer of anti-DNP antibodies in BALB/c mice out of three lipopeptide constructs incorporating the different T_{H} epitopes that were tested, lipidated DNP-T_{H(MV)} can only induce moderate antibody production in C57/B6 mice compared to DNP-_{T(ova)}. It was also demonstrated that mixing a construct incorporating a T_{H} epitope that induced low antibody production with one incorporating a T_{H} epitope that could induce higher secondary anti-DNP antibody production could elevate levels of antibody titre to a point where it was not statistically different to that of the better antibody inducing lipopeptide construct in both strains of mice.

### Example 4

### Determining the Specificity of Anti-DNP Antibodies Using DNP Derivatives

Inhibition ELISAs were utilised to determine whether antibody-titres detected in previous direct-binding ELISAs consisted mainly of anti-DNP antibodies. All immunogens (peptide- and protein-based) incorporated the 2,4-DNP isomer.

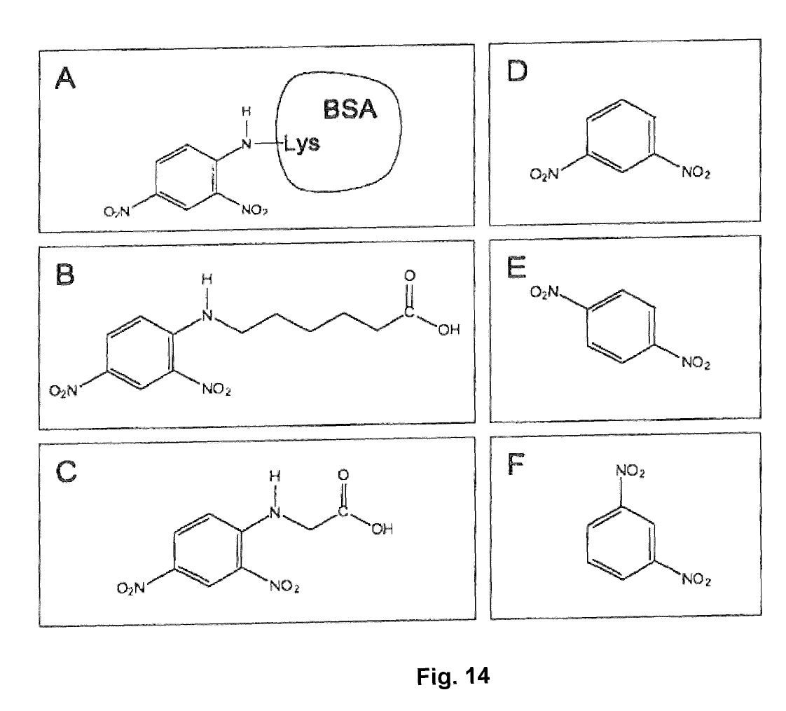

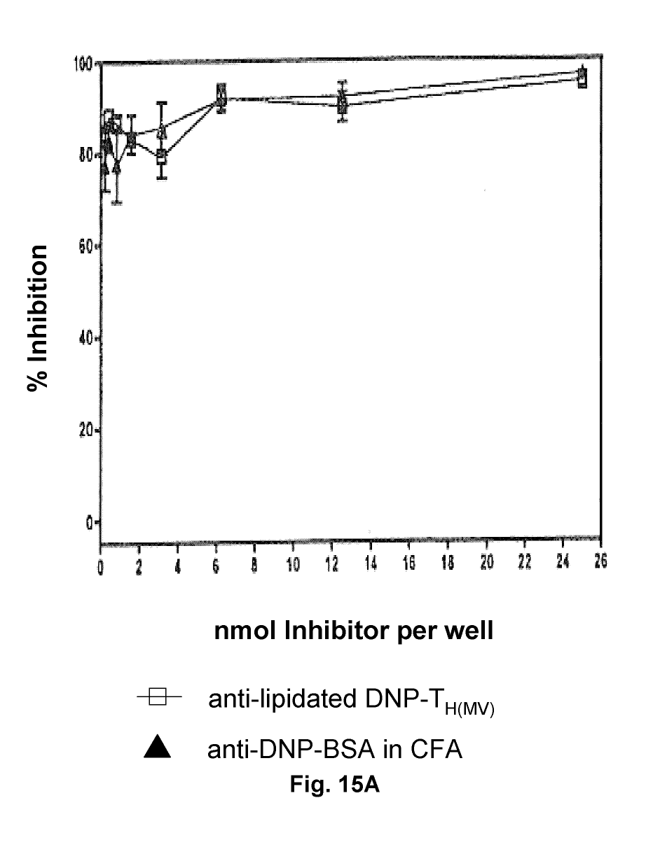

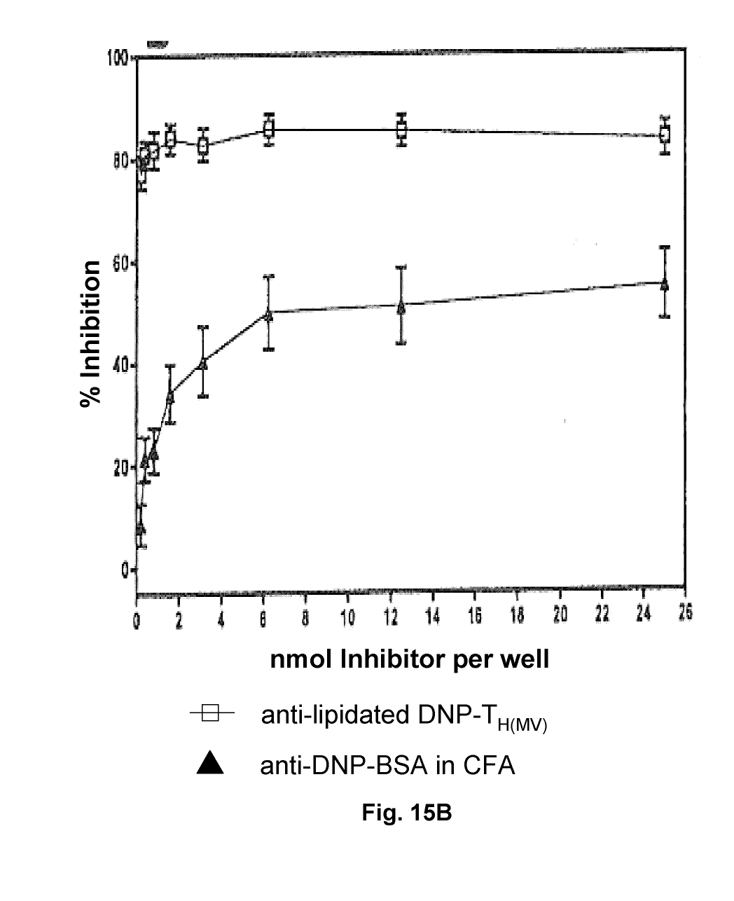

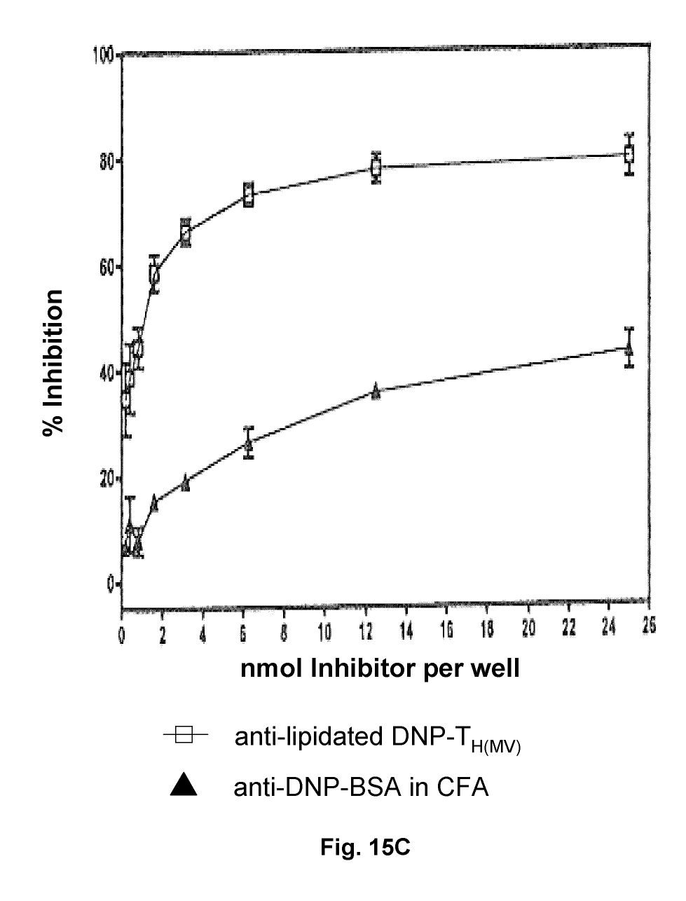

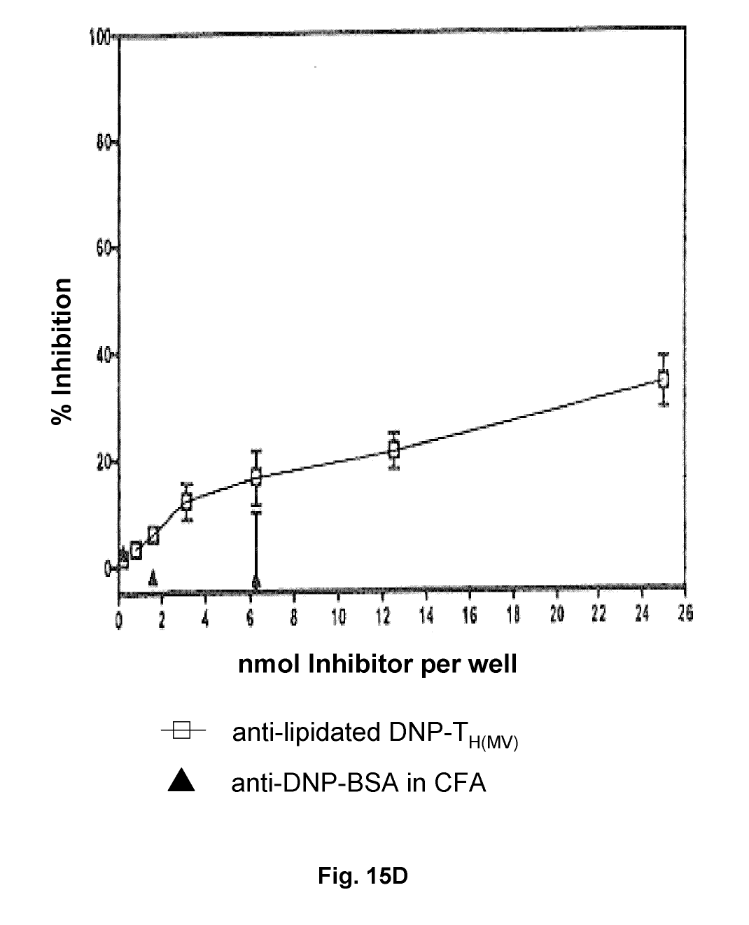

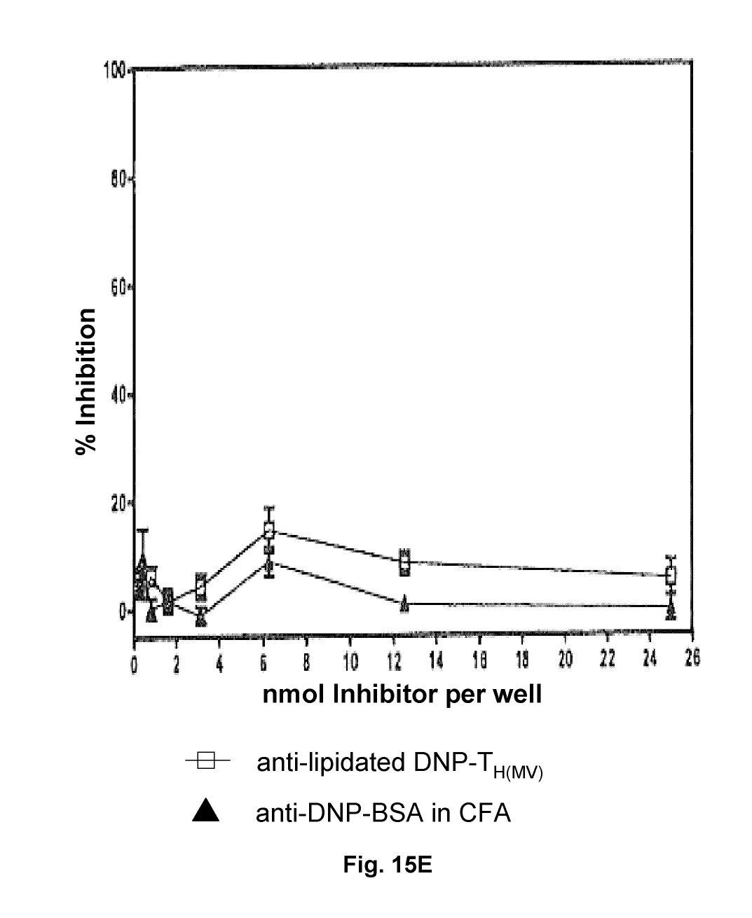

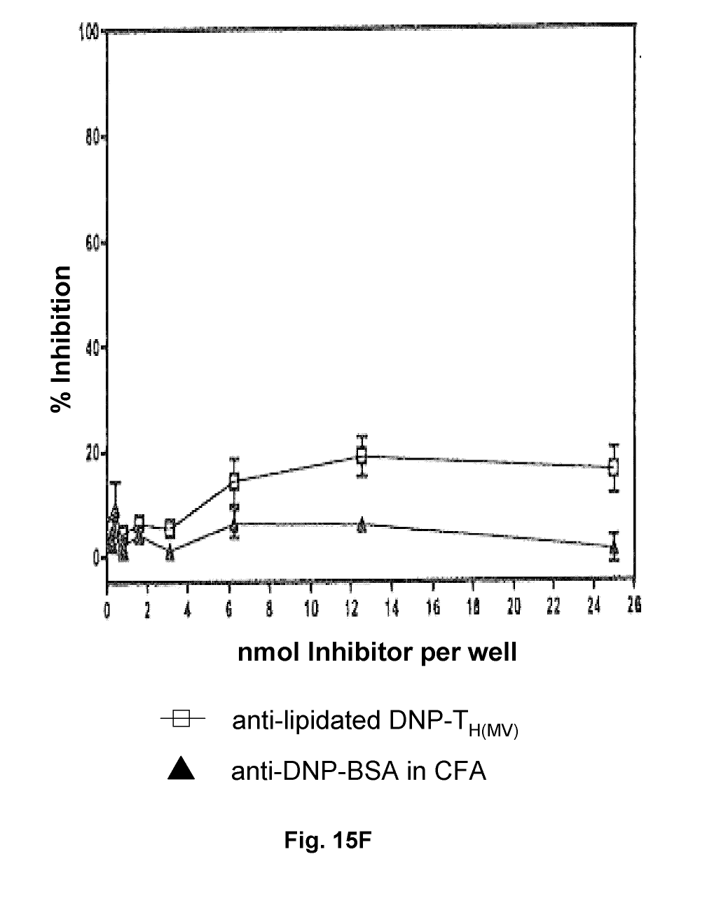

Sera from two BALB/c groups displaying the highest antibody titres (immunized with either the positive control; DNP-BSA administered in CFA or the test immunogen; lipidated DNP-T_{H(MV)}) were assessed. Sera were incubated with different amounts of various inhibitors (FIG. 14) before addition to DNP-BSA coated plates. As expected, sera from both groups were completely inhibited with DNP-BSA (FIG. 15, panel A). When 2,4-DNP-ahx (the DNP-derivative used to couple to all DNP-peptide immunogens) was used as an inhibitor (FIG. 15, panel B), approximately 80% inhibition was observed for the lipidated DNP-TH(MV) sera, whereas only a maximum of approximately 55% was observed for DNP-BSA sera. 2,4-DNP-glycine was also used as an inhibitor (FIG. 15, panel C), once again, a high level of inhibition (80%) was observed for the peptide construct, and lower of level of inhibition for the protein vaccine. These ELISAs confirm that antibody elicited by lipidated DNP-TH(MV) vaccination are targeting the DNP molecule.

### Example 5

### Determining the Fine Specificity of Anti-DNP Antibodies by Using DNP Isomers as Inhibitors

Different isomers (2,4-DNP, 2,5-DNP and 2,6-DNP) were also used to determine the fine specificity of the antibodies (FIG. 14, panels D-F). Antibody induced by lipidated 2,4-DNP-T_{H(MV)} was inhibited by 2,4-DNP (35%) at the highest concentration examined, and to a lower degree (<20%) by 2,6-DNP, indicating a high degree of specificity for 2,4-DNP. No inhibition of binding by any of the DNP isomers was observed in sera induced by the protein carrier DNP-BSA administered in CFA. Irrelevant inhibitors tyrosine and phenol (both possessing phenol rings) were also tested, showing no inhibition.

### Example 6

### Synthesis and Immunological Study of Peptide-Based Amphetamine, Norcocaine and Morphine Vaccines as for a Vaccine for Prevention and/or Therapy of Drug Addiction

### Preparation of Amphetamine-BSA (Amphetamine-BSA)

Amphetamine was conjugated to BSA using carbodiimide (FIG. 6) before extensive dialysis. The substitution ratio was determined by UV spectrometry using the molar extinction coefficients of BSA (41,383, 22,770, and 20,648 M⁻¹cm⁻¹) and amphetamine succinamide (37,209,193 M⁻¹ cm⁻¹) at the 3 wavelengths of 280 nm, 257 nm and 252 nm respectively, as described by Mongkolsirichaikul et al. [Journal of Immunology Methods 157(1-2):189-95, 1993]. It was found that approximately 29 amphetamine molecules were attached to each BSA molecule through the carboxyl group present on the side chains of aspartic acid and glutamic acid residues of which BSA has 40 and 59 residues, respectively.

### Preparation of Norcocaine-BSA (Cocaine-BSA)

Succinyl norcocaine (FIG. 7) was conjugated to BSA in a reaction mixture adjusted to pH 8 before dialysis. The substitution ratio was unable to be determined because a molar extinction coefficient for norcocaine was not available.

### Preparation of Completely Synthetic Peptide-Based Drug-Vaccines

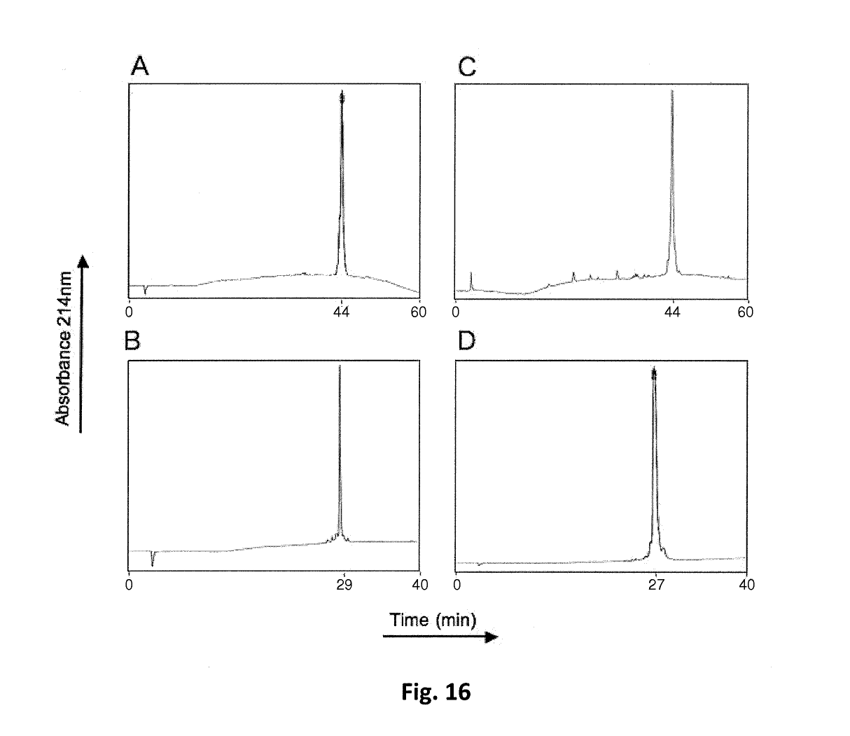

Succinyl amphetamine and succinyl norcocaine were coupled to peptide constructs (FIGS. 4 and 5) using the same method as that used for the coupling of amino acid during peptide synthesis. Similarly, succinyl morphine was coupled to the peptide construct to generate a lipopeptide vaccine containing morphine. Analytical chromatograms of purified peptide constructs are shown in FIG. 16, with Table 3 displaying the masses of peptide and lipopeptide constructs as determined by ESI-MS.

### Evaluation of the Ability of the Peptide-Based Amphetamine Constructs to Induce Specific Anti-Amphetamine Antibodies

Groups of mice were administered with the lipidated amphetamine-T_{H(MV)} in saline, and the non-lipidated amphetamine-T_{H(MV)} in either saline or CFA. Amphetamine-BSA was also inoculated either in CFA or saline. Because of its small size, amphetamine is not able to coat ELISA plates directly. Thus several different peptide constructs were used to coat ELISA plates to determine an optimal coating antigen for ELISA. First, the immunogens that were administered in mice, amphetamine-BSA and amphetamine-T_{H(MV)} were used as antigen to coat plates.

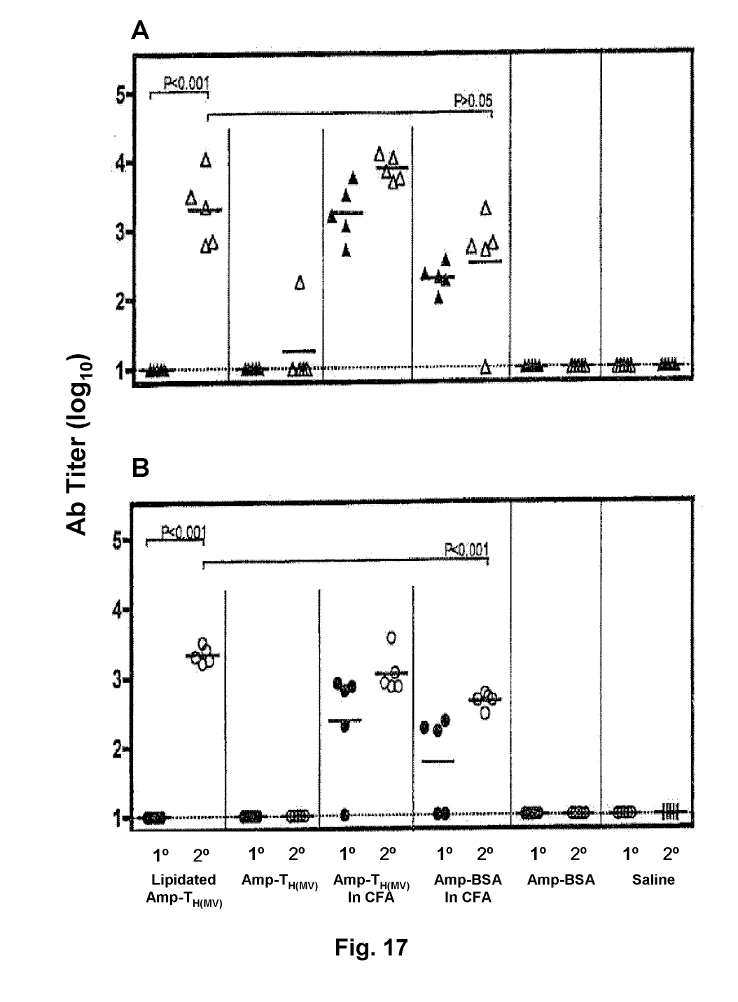

Higher antibody titres in sera obtained from mice that had received amphetamine-T_{H(MV)} constructs were detected on the amphetamine-T_{H(MV)} coated plate (FIG. 17, panel A), possibly due to the presence of anti-T_{H(MV)} antibodies. At the same time, amphetamine-BSA administered in CFA induced sera displayed higher antibody titres on amphetamine-BSA coated plates (FIG. 17, panel B), possibly due to the presence of anti-BSA antibodies.

TABLE 3
Theoretical and actual mass in Daltons of amphetamine-TH,
cocaine-TH and morphine-TH constructs as determined
by ESI-MS

| | Theoretical | Observed |
|---|---|---|
| Construct | mass | mass |
| Lipidated amphetamine-T_{H(MV)} | 3,029.89 | 3,030.37 |
| Non-lipidated amphetamine-T_{H(MV)} | 2,201.69 | 2,201.09 |
| Non lipidated amphetamine-T_{H(Flu)} | 2,061.59 | 2,061.47 |
| Non-lipidated amphetamine-T_{H(ova)} | 2,117.79 | 2,118.15 |
| Lipidated cocaine-T_{H(Mv)} | 3,184.25 | 3,184.04 |
| Non-lipidated cocaine-T_{H(MV)} | 2,355.75 | 2,356.97 |
| Non-lipidated cocaine-T_{H(Flu)} | 2,215.35 | 2,216.20 |
| Lipidated morphine-T_{H(OVA)} | 3,095.3 | 3,096.3 |

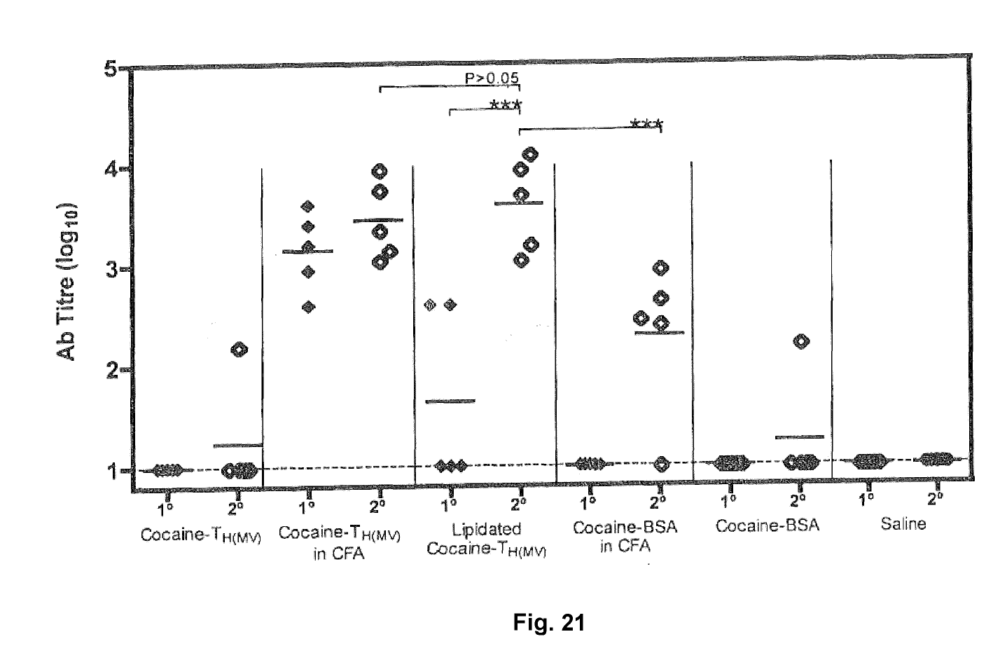

In attempt to find an ELISA plate coating antigen with an irrelevant T_{H} epitope to reduce the detection of anti-T_{H} antibodies, amphetamine-T_{H(Flu)} and amphetamine-T_{H(ova)} were used to coat ELISA plates. It was found that similar antibody titres were detected in the sera of mice immunised with the test immunogens on the plates which were coated with either amphetamine-T_{H(Flu)} or amphetamine-T_{H(ova)} (FIG. 21, panels C and D).

It was shown in all ELISAs that there was a significant increase from primary to secondary antibody titre for mice immunised with lipidated amphetamine-T_{H(Mv)}. Furthermore, the ELISA coated with the irrelevant T_{H} epitope (amphetamine-T_{H(ova)}, showed significantly higher antibody titres for the group administered lipidated amphetamine-T_{H(MV)} compared to the group given the amphetamine-BSA administered in CFA positive control. Overall, the data showed that anti-amphetamine antibodies were induced by the peptide- and protein-based immunogens. Due to the excellent reproducibility of the ELISA performed on amphetamine-T_{H(ova)}, it was chosen for further amphetamine ELISA studies.

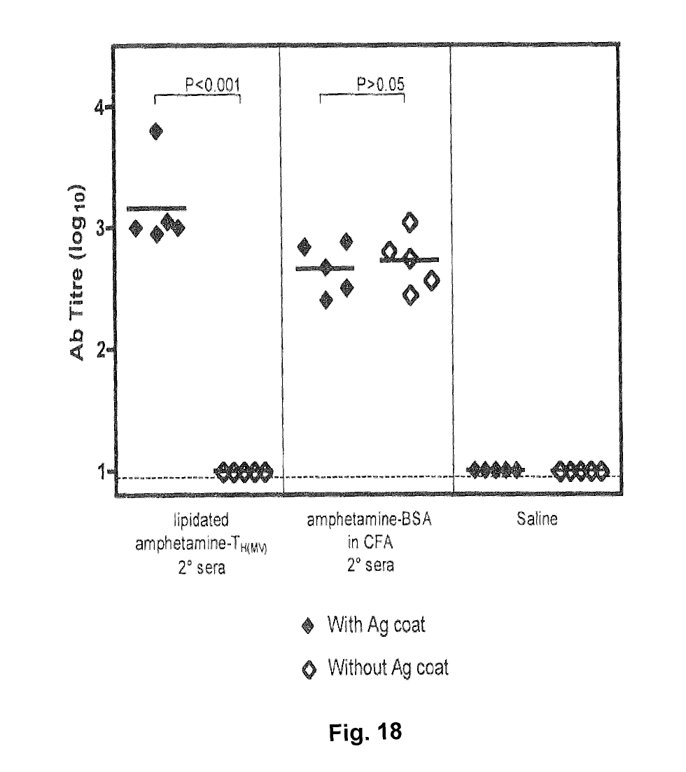

Due to some antibody binding observed on non-antigen coated control wells by sera induced with amphetamine-BSA administered in CFA (data not shown), it was postulated that anti-BSA antibodies were present. This hypothesis was proven in a subsequent ELISA (FIG. 18), where it was demonstrated that in the absence of an amphetamine construct antigen coat, antibody induced by amphetamine-BSA administered in CFA but not lipidated amphetamine-TH(Mv) could still bind BSA-coated wells.

### Example 6

### Determining the Specificity of Anti-Amphetamine Antibodies

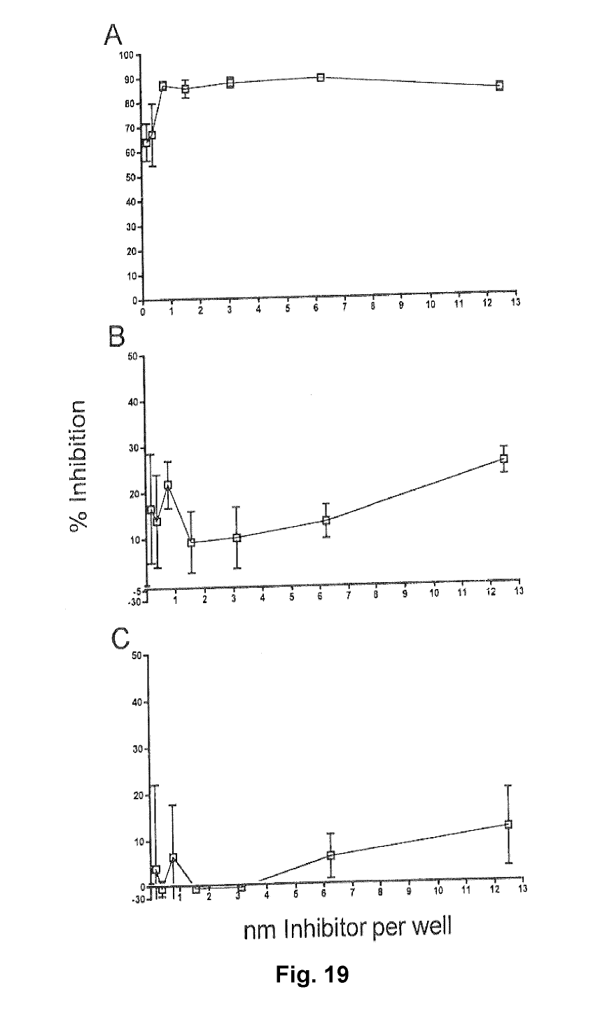

A competitive ELISA utilising various inhibitors was performed to assess the specificity of the anti-amphetamine antibodies (FIG. 19). Secondary sera from mice administered with the lipidated-amphetamine-T_{H(MV)} construct were tested in this study, with amphetamine-T_{H(ova)} used as coating antigen to avoid the binding of anti-T_{H(MV)} antibody present in the sera. In addition to amphetamine, amphetamine-T_{H(ova)} was used as an inhibitor to determine maximum percentage inhibition of the immunogen. Another peptide, amphetamine-T_{H(Flu)} was included as an inhibitor to give more information about the specificity of anti-amphetamine antibodies. The results show that as expected, a high level of inhibition was achieved when amphetamine-T_{H(ova)} was used as an inhibitor (FIG. 18, panel A). Amphetamine-sulfate was also tested, which displayed a low level of inhibition at the concentrations we used (FIG. 19, panel C).

Although it was expected that amphetamine-T_{H(Flu)} would display a similar level of inhibition of binding to amphetamine-T_{H(ova)}, this was not observed. A lower level of inhibition was observed for the same concentration of inhibitor compared to when amphetamine-T_{H(ova)} was used (FIG. 19, panel B). This suggests that the 3-dimensional conformation of amphetamine-T_{H(ova)} changes when in solution as opposed to when it was used as an ELISA plate coating antigen and immobilised onto a solid phase, shielding the amphetamine group from antibody binding. Methamphetamine hydrochloride was tested for possible cross reactivity and cocaine hydrochloride was selected as an irrelevant inhibitor. No inhibition was observed for either of these drugs.

### Example 7

### Intranasal Amphetamine Study

Completely synthetic self-aduvanting lipopeptide vaccines have the advantage of being able to be delivered intranasally. An intranasal amphetamine-vaccine study was performed to assess the induction of not only total, but also mucosal (IgA) antibodies by lipidated amphetamine-T_{H(Mv)}. Negative controls (non-adjuvanted amphetamine-T_{H(Mv)}, amphetamine-BSA and mock immunization by saline) were also administered to mice by the i.n. route, and secondary sera and saliva were collected 7 and 8 days after the boost respectively.

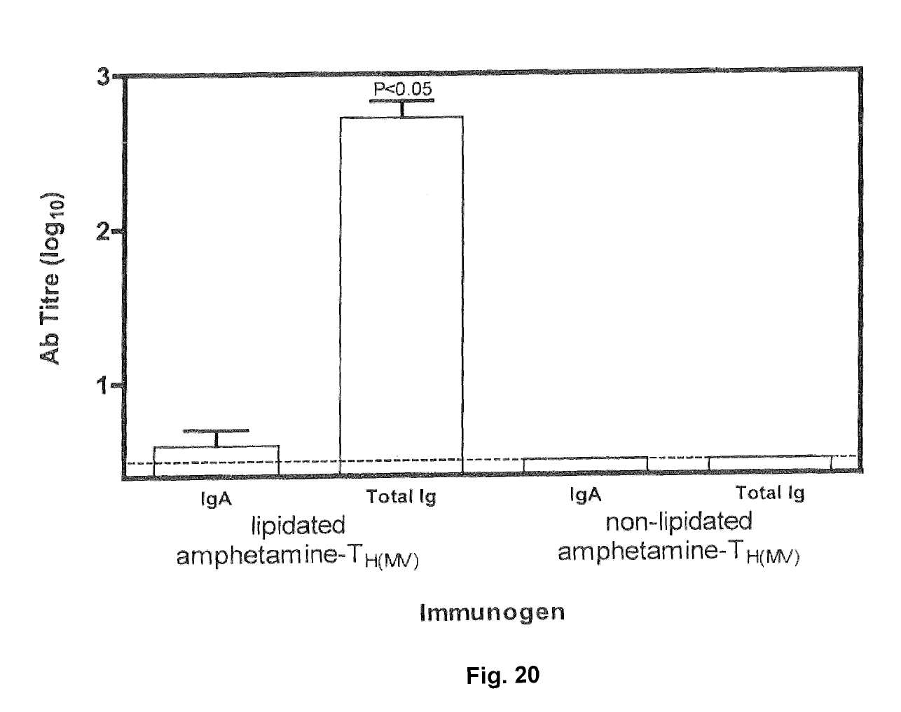

Although anti-amphetamine antibodies were not detected in saliva (data not shown), significant levels of anti-amphetamine antibodies (P<0.05) were present in the in the secondary response of sera intranasally inoculated with lipidated amphetamine-T_{H(Mv)} (FIG. 20). However, no detectable level of anti-amphetamine IgA antibodies was found in the sera.

### Example 8

### Detection of Anti-Cocaine Antibodies by Direct Binding ELISA

The immunogenicity of self-adjuvanting peptide-based cocaine vaccines was also tested. Using cocaine-T_{H(Flu)} as a coating antigen, it was shown in FIG. 21, that the secondary antibody titer induced by lipidated cocaine-T_{H(Mv)} was significantly increased from the primary response (P<0.001). It was also shown to be higher than the secondary response (P<0.001) elicited by the adjuvanted protein-carrier based cocaine vaccine (cocaine-BSA administered in CFA). This may indicate that the lipopeptide construct was superior in terms of immugenicity to cocaine-BSA. However, it was not possible to determine the substitution rate of norcocaine to BSA. Consequently, a direct comparison of the immunogenicity between peptide- and protein-carrier based vaccines could not be made.

### Example 9

### Detection of Anti-Morphine Antibodies by Inhibition ELISA

Mice were inoculated with 6-succinyl-morphine-Lys (Pam₂CysSer₂)-T_{H} (lipidated-morphine-T_{H(OVA)}) (see FIG. 22) on day 0 and day 21, with the mice being bled on days 21 and 31. Sera was prepared from both bleeds.

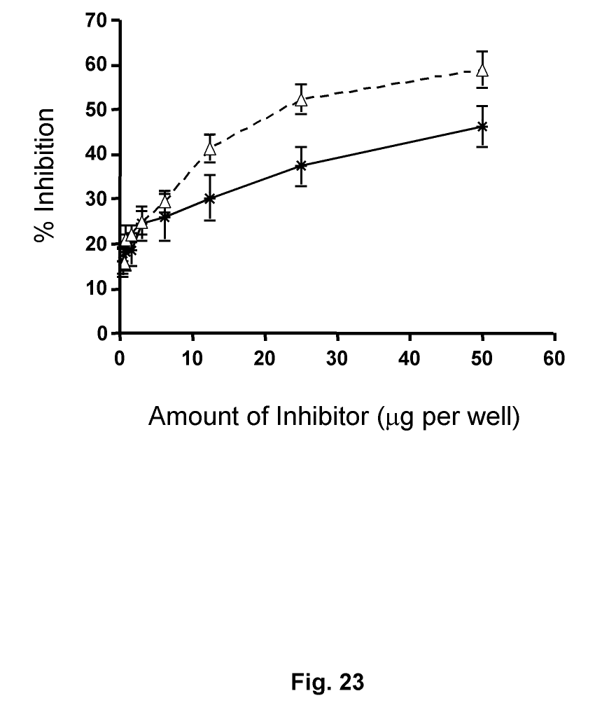

Inhibition ELISAs were performed on the day 31 sera. The sera were incubated for 90 minutes at room temperature with either 6-succinyl-morphine-Lys (Pam₂CysSer₂)-T_{H} (lipidated-morphine-T_{H(OVA)}) (see FIG. 23; broken line) or Cys-Lys(Pam₂CysSer₂)-T_{H} (lipidated-T_{H(OVA)}) as the inhibitors or no inhibitor. The sera/inhibitors were then added to wells coated with the lipidated-morphine-T_{H(OVA)}.

Percentage inhibition was calculated using the formula:

$$\% inhibition = ( 1 - \frac{\begin{matrix}Absorbance ( with inhibitor ) - \\ background\end{matrix}}{\begin{matrix}Absorbance ( without inhibitor ) - \\ background\end{matrix}} ) \times 100 \%$$

As seen from FIG. 23, inhibition is greater in the presence of sera obtained from animals inoculated with 6-succinyl-morphine-Lys (Pam₂CysSer₂) indicating that antibodies against the morphine moiety have been generated.

As will be recognised by those skilled in the art, other derivatives of the drugs and other methods can be used to attach the drug to the lipopeptide and to a protein. For example, these could include bromoacetylated butylamino methamphetamine and norcocaine constructs, as well as derivatives of benzoyl ecgonine in the case of cocaine. Succinyl morphine may also be used.

Those skilled in the art will appreciate that the invention described herein is susceptible to variations and modifications other than those specifically described. It is to be understood that the invention includes all such variations and modifications. The invention also includes all of the steps, features, compositions and compounds referred to, or indicated in this specification, individually or collectively, and any and all combinations of any two or more of said steps or features.

### BIBLIOGRAPHY

Alexander et al. Immunity 1:751-761, 1994
Berzofsky et al. J Clin Invest 88:876-884, 1991
Berzofsky, In: “The Year in Immunology, Vol. 2” page 151, Karger, Basel, 1986
Dawson et al. Science 266:243-247, 1994
Ghosh et al. Immunol 104:58-66, 2001
Goodman and Sercarz Ann Rev Immunol 1:465, 1983
International Patent Publication No. WO 00/46390
Jackson et al. Virol 98:613-623, 1994
Jones et al. Xenobiotica 5:155, 1975
Livingstone and Fathman Ann Rev Immunol 5:477, 1987
Metzger et al. Int J Pept Protein Res 55:545, 1991
Metzger et al. J Pept Sci 1:184, 1995
Mongkolsirichaikul et al. Journal of Immunology Methods 157(1-2):189-95, 1993
Muhlradt et al. Infect Immun 66:4804, 1998
Muhlradt et al. J Exp Med 185:1951, 1997
Muir et al. Proc Natl Acad Sci USA 95:6705-6710, 1998
Muller et al. Mol Immunol 32:37-47, 1995
Panina-Bordignon et al. Eur J Immun 19: 2237-2242, 1989
Partidos et al. J Gen Virol 71:2099-2105, 1990
Sacht et al. Eur J Immunol 28:4207, 1998
Tarn et al. Biopolymers (Peptide Science) 51:311-332, 1999
United States Patent Application No. 0020018806
U.S. Pat. No. 5,700,910
U.S. Pat. No. 6,024,964
Wiesmuller et al. Hoppe Seylers Zur Physiol Chem 364:593, 1983
Winzler et al J Exp Med 185, 317, 1997
Yokoyama et al. Molecular Immunology 29(7-8):935-947, 1992
Zeng et al. J Pept Sci 2:66, 1996

What is claimed is:

1. A lipopeptide comprising a lipid moiety, a T-helper (T_{H}) epitope, a target epitope specific for a drug of dependence and linker moiety, wherein the linker moiety comprises at least a first, second and third reactive site and wherein lipid moiety is covalently linked to the first reactive site, the T_{H} epitope is covalently linked to the second reactive site and the target epitope is covalently linked to the third reactive site.

2. The lipopeptide of claim 1, wherein the linker is an amino acid or other tri-functional moiety.

3. The lipopeptide of claim 2, wherein the amino acid is selected from the group consisting of aspartic acid, glutamic acid and analogs thereof.

4. The lipopeptide of claim 2, wherein the amino acid is selected from the group consisting of lysine, ornithine, diaminopropionic acid, diaminobutyric acid, and analogs thereof.

5. The lipopeptide of claim 4, wherein the linker moiety is lysine and the T_{H} epitope attached to the carboxyl group, the target epitope is attached to α-amino group and the lipid moiety is attached to the ε-amino group.

6. The lipopeptide of claim 4, wherein the linker moiety is lysine and the target epitope is attached to the carboxyl group, the T_{H} epitope is attached to the α-amino group and the lipid moiety is attached to the ε-amino group.

7. The lipopeptide of claim 4, wherein the linker moiety is lysine and the lipid moiety is attached to the carboxyl group, the T_{H} epitope is attached to the α-amino group and the target epitope is attached to the ε-amino group.

8. The lipopeptide of claim 1, wherein the lipid moiety is selected from the group consisting of palmitoyl, stearoyl and decanoyl.

9. The lipopeptide of claim 1, wherein the lipid moiety is a molecule having a structure of Formula (II):

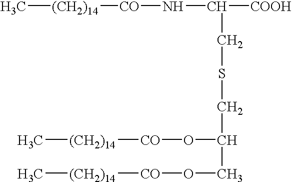

10. The lipopeptide of claim 9, wherein the lipid moiety is N-palmitoyl-S-[2,3-bis(palmitoyloxy)propyl]cysteine.

11. The lipopeptide of claim 1, wherein the lipid moiety is a molecule having a structure of Formula (III):

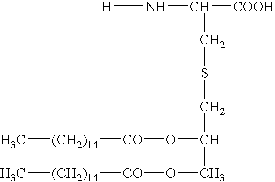

12. The lipopeptide of claim 11, wherein the lipid moiety is S-[2,3-bis(palmitoyloxy)propyl]cysteine.

13. The lipopeptide of claim 1, wherein the lipid moiety is a molecule having a structure of Formula (IV): 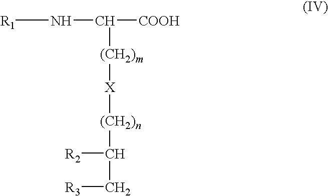
wherein:
(i) X is selected from the group consisting of sulfur, oxygen, disulfide (—S—S—), and methylene (—CH₂—), and amino (—NH—);
(ii) m is an integer being 1 or 2;
(iii) n is an integer from 0 to 5;
(iv) R₁ is selected from the group consisting of hydrogen, carbonyl (—CO—), and R′—CO— wherein R′ is selected from the group consisting of alkyl having 7 to 25 carbon atoms, alkenyl having 7 to 25 carbon atoms, and alkynyl having 7 to 25 carbon atoms, wherein said alkyl, alkenyl or alkynyl group is optionally substituted by a hydroxyl, amino, oxo, acyl, or cycloalkyl group;
(v) R₂ is selected from the group consisting of R—CO—O—, R—O—, R—O—CO—, R′—NH—CO—, and R—CO—NH—, wherein R′ is selected from the group consisting of alkyl having 7 to 25 carbon atoms, alkenyl having 7 to 25 carbon atoms, and alkynyl having 7 to 25 carbon atoms, wherein said alkyl, alkenyl or alkynyl group is optionally substituted by a hydroxyl, amino, oxo, acyl, or cycloalkyl group; and
(vi) R₃ is selected from the group consisting of R—CO—O—, R′—O—, R′—O—CO—, R′—NH—CO—, and R—CO—NH—, wherein R′ is selected from the group consisting of alkyl having 7 to 25 carbon atoms, alkenyl having 7 to 25 carbon atoms, and alkynyl having 7 to 25 carbon atoms, wherein said alkyl, alkenyl or alkynyl group is optionally substituted by a hydroxyl, amino, oxo, acyl, or cycloalkyl group,
and wherein each of R₁, R₂ and R₃ is the same or different.

14. The lipopeptide of claim 13, wherein the lipid moiety is a chiral molecule, wherein the carbon atoms directly or indirectly covalently bound to integers R₁ and R₂ are asymmetric dextrorotatory or levorotatory configuration.

15. The lipopeptide of claim 13, wherein X is sulfur; m and n are both 1; R₁ is selected from the group consisting of hydrogen, and R′—CO—, wherein R′ is an alkyl group having 7 to 25 carbon atoms; and R₂ and R₃ are selected from the group consisting of R′—CO—O—, R′—O—, R′—O—CO—, R′—NH—CO—, and R—CO—NH—, wherein R′ is an alkyl group having 7 to 25 carbon atoms.

16. The lipopeptide of claim 13, wherein R′ is selected from the group consisting of: palmitoyl, myristoyl, stearyl and decanol. More preferably, R is palmitoyl.

17. The lipopeptide of claim 1, wherein the lipid moiety is a molecule having a structure of Formula (V):

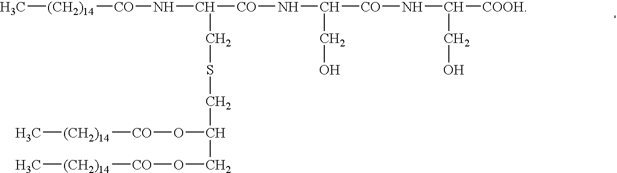

18. The lipopeptide of claim 1, wherein the lipid moiety is a molecule having a structure of Formula (VI):

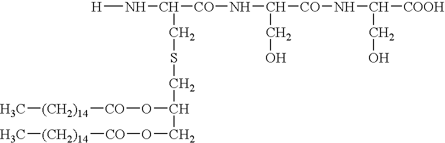

19. The lipopeptide of claim 1, wherein the target of dependence is a lipophilic drug of dependence.

20. The lipopeptide of claim 1, wherein the drug of dependence is selected from the group consisting of MA, MDMA, cocaine, cannabis, morphine, nicotine and their derivatives.

21. The lipopeptide of claim 1, wherein the T_{H} epitope comprises an amino acid sequence selected from list consisting of SEQ ID NO:1, SEQ ID NO:2 and SEQ ID NO:3.

22. A method of eliciting an antibody response to a drug of dependence in a subject, said method comprising administering to said subject a lipopeptide according to claim 1.

23. A method for treating an addiction to a drug of dependence in a subject, the method comprising administering to a subject a lipopeptide according to claim 1.

24. A method of manufacture of a medicament for the treatment or prevention of drug dependency, wherein said medicament contains a lipopeptide according to claim 1.
